(19) United States
(12) Patent Application Publication
(10) Pub. No.: US 2026/0139018 A1
(43) Pub. Date: May 21, 2026

(54) MODULATION OF WNT SIGNALLING IN CORNEAL DISORDERS

(71) Applicant: Surrozen Operating, Inc., South San Francisco, CA (US)

(72) Inventors: Priya HANDA, San Bruno, CA (US); Sungjin LEE, Palo Alto, CA (US); Yang LI, Mountain View, CA (US); Laura SANMAN, South San Francisco, CA (US); Geertrui VANHOVE, Menlo Park, CA (US); Wen-Chen YEH, Belmont, CA (US)

(21) Appl. No.: 18/876,603

(22) PCT Filed: Jun. 16, 2023
(86) PCT No.: PCT/US2023/068623

§ 371 Date: Dec. 18, 2024

Related U.S. Application Data
(60) Provisional application No. 63/353,968, filed on Jun. 21, 2022.
(60) Provisional application No. 63/469,499, filed on May 29, 2023.

(51) Int. Cl. C07K 14/47; A61K 38/00; A61P 27/02; C07K 16/28; C12N 5/079

(52) U.S. Cl. CPC C07K 14/4705; A61P 27/02; C07K 16/2863; C12N 5/0621; A61K 38/00; C07K 2319/00; C12N 2501/415

(57) ABSTRACT

Current treatments for corneal disorders, also called keratopathies, depend on the stage of the keratopathy. For mild keratopathies, supportive treatments such as saline eyedrops may be sufficient to ameliorate symptoms. The present disclosure provides methods of treating corneal disorders with modulators of the WNT signaling pathway. More particularly, provided are treatments for cornea epithelial and/or endothelial injuries, defects, deficiencies, and dystrophies. Further provided are methods of dosing and pharmaceutical compositions for the treatment of corneal disorders.

### CROSS REFERENCE TO RELATED APPLICATIONS

This application is a National Stage application under 35 U.S.C. § 371 of International Application No. PCT/US2023/068623, filed Jun. 16, 2023, which claims the benefit under 35 U.S.C. § 119(e) of U.S. Provisional Application No. 63/353,968, filed Jun. 21, 2022, and U.S. Provisional Application No. 63/469,499, filed May 29, 2023, the entire contents of which are incorporated herein by reference.

### SEQUENCE LISTINGS

The Sequence Listing XML associated with this application is provided in XML file format and is hereby incorporated by reference into the specification. The name of the XML file containing the Sequence Listing XML is SRZN_021_03WO_ST26.xml. The XML file is 21,937 bytes, and created on Jun. 10, 2023, and is being submitted electronically via USPTO Patent Center.

### FIELD OF THE INVENTION

The disclosure provides WNT signal modulators to treat various corneal disorders. More particularly, provided are treatments for cornea epithelial and/or endothelial injuries, defects, deficiencies, and dystrophies.

### BACKGROUND

The vertebrate cornea is an optically clear layered tissue representing the first and strongest refractive medium of the eye's anatomy. (Patel & Tutchenko, Contact Lens & Ant. Eye, 2019 October 42(5), 575-80.) The cornea must remain sturdy to contain and protect the eye; transparent to allow visible light to pass into the eye; and appropriately shaped, symmetrical, and smoothed to properly refract and direct a sharp image to the image-forming retina tissue at the back of the eye.

Unfortunately, the cornea is susceptible to injury and disease that cause scarring, discoloration, or worse, which can severely impair vision and quality of life. Current treatments for corneal disorders, also called keratopathies, depend on the stage of the keratopathy. For mild keratopathies, supportive treatments such as saline eyedrops may be sufficient to ameliorate symptoms. For severe keratopathies, cornea transplant or cellular transplant may be necessary because currently there are no non-surgical treatment options available. Cornea and cellular transplants, i.e., keratoplasties, are costly, invasive, and risk tissue rejection. Further, available supply of donor cornea and cells is often lacking.

There may be a promising treatment avenue: signaling of the WNT developmental pathway has been implicated as important in the stratification and layering of mammalian cornea. (Zhang et al., Development, 2015 142:3383-93.) WNT signaling proteins are among the most important developmental signaling molecules in the animal kingdom. In animals ranging from fruit fly to mouse to frog to nematode worm to flatworm, involvement of WNT proteins has been demonstrated in body segmentation, central nervous system (CNS) patterning, dorsal/ventral axis formation, and much more. (See, e.g., Wodarz & Nusse, Annu. Rev. Cell Dev. Biol., 1998 14:59-88.)

Mutations and modulations of WNT proteins are also thought to play significant roles in numerous cancers and dystrophies. (Nusse, Cell Res., 2005 (15), 28-32.) In the context of the cornea, it is unknown whether WNT modulation post-developmentally may lead to improvement in keratopathies (disorders of the cornea) and injuries. WNT signaling promotes growth and sternness of cornea endothelial and epithelial cells in vitro (Maurizi et al. Sci. Rep., 2020; Nakatsu et al Invest. Opthalm. Vis. Sci. 2011), indicating that activating Wnt signaling may have a therapeutic effect in keratopathies where cornea cells are deficient. Wnt signaling is also central to fibrosis and elevated in corneal scarring (Chawla & Ghosh, J. Cell Physiol. 2017), indicating that antagonizing Wnt signaling may have a therapeutic effect in keratopathies involving scarring, fibrosis, EMT, and EnMT. (Kawashima et al. Mol. Vis., 2013). Understanding the keratopathy disease progression and the WNT signal involvement extends to the possibilities of new treatments.

Furthermore, cornea tissue is one of several rare and valuable organ donor tissues. Cornea transplants may be used to treat numerous keratopathies. However, donated corneas may not grow or heal properly in the recipient subject. Accordingly, it would be desirable to have a treatment that would promote ex vivo cell growth in donated cornea tissue. Additionally, treatment could also occur post-transplant to increase the survival of transplanted tissue in the recipient.

Understanding keratopathy disease progression and the involvement of WNT signaling in this progression could extend possibilities of new treatments. The present disclosure provides methods of modulating WNT signaling to treat corneal epithelial and endothelial disorders and to promote ex vivo cell growth and maintenance of corneal epithelial and endothelial tissues and cells.

### BRIEF SUMMARY

The present disclosure is directed to WNT signaling modulators to treat various corneal disorders. More particularly, the present disclosure provides treatments for various keratopathic indications, including, e.g., cornea epithelial and/or endothelial injuries, defects, deficiencies, and dystrophies.

In some embodiments, the present disclosure provides a method of treating a subject suffering from a keratopathy or corneal injury comprising administering the subject, an engineered WNT signaling modulator. In certain embodiments, the WNT signaling modulator is an engineered WNT agonist or an engineered WNT antagonist. In further embodiments the engineered WNT agonist and WNT antagonist comprise binding compositions (or regions) that bind to one or more Fzd receptors and binding compositions (or regions) that bind to one or more LRP receptors. In further embodiments, the binding compositions of the engineered WNT agonist are selected from the group consisting of: a Fzd binding composition, a Lrp5 binding composition, a Lrp6 binding composition, or a LRP5/6 binding composition. In particular embodiments, the FZD binding composition of the engineered WNT agonist binds to one or more Fzd receptor selected from the group consisting of Fzd1, Fzd2, and Fzd7.

In some embodiments, the engineered WNT agonist or WNT antagonist are administered independently at early and/or late stages of keratopathy. In alternative embodiments, the WNT agonist and WNT antagonist are administered sequentially at early and/or late stages of keratopathy, or the WNT agonist and WNT antagonist are co-administered at early and/or late stages of keratopathy. In further embodiments, the WNT agonist is administered before or after the WNT antagonist.

In an aspect, the present disclosure provides a method of treating a keratopathy in a subject, comprising administering a WNT signaling modulator to the subject. In another embodiment, the present disclosure provides a method comprising administering the isolated polynucleotide encoding a polypeptide of the molecule, wherein the polynucleotide is optionally an mRNA, optionally a modified mRNA, or administering an expression vector comprising the isolated polynucleotide.

In preferred embodiments, the subject is a human patient. In some embodiments, the WNT signaling modulator may be an engineered WNT signaling modulator. In some embodiments, the WNT signaling modulator may be an engineered WNT agonist or an engineered WNT antagonist.

In a preferred embodiment, the engineered WNT agonist is selected from: (i) a WNT3a; (ii) a WNT mimetic; or (iii) an R-spondin mimetic or (iv) a WNT mimetic fused to a mutant R-spondin mimetic, referred to as a superSWAP or superagonist. See, e.g., PCT Application Publication No. WO 2021173726, which discloses non-limiting examples of superSWAP molecules. In a still preferred embodiment, the WNT mimetic is a SWAP™ compound, and the R-spondin mimetic is a SWEETS™ compound; and the molecule that encompasses the SWAP and the SWEETs in one combined molecule is referred to as superSWAP or superagonist. Particularly, the WNT signaling modulator may be a canonical WNT modulator or a non-canonical WNT modulator (see, e.g., Ackers and Malgor (2017) Diabetes & Vascular Res., 15:3-13).

In any embodiment of the method, the WNT signaling modulator may be co-administered with R-spondin, or any R-spondin homologue, mutant, fragment, mimic, or any combination thereof.

In any embodiment of the method, the wnt signaling modulator may be a WNT mimetic fused to a mutant Rspodin mimetic referred to here as superSWAP or superagonist.

In preferred embodiments, the WNT signaling modulator is administered to the subject in a therapeutically effective dose. In some embodiments, the WNT signaling modulator may be administered via eye drop, hydrogel, depot, or intraocular injection.

In any embodiment described herein, the WNT signaling modulator may be suspended in aqueous solution. The WNT signaling modulator may be suspended in aqueous solution at a concentration of about 0.01 pM, 0.1 pM, 1 pM, 0.01 nM, 0.1 nM, 1 nM, 10 nM, 100 nM, 1 μM, 10 μM, 100 μM, 1 mM, 10 mM, 100 mM, up to about 1 μM. In preferred embodiments, the WNT signaling modulator is suspended in aqueous solution at a concentration ranging from about 0.1 nM to 1 μM. In still preferred embodiments, the WNT signaling modulator is suspended in aqueous solution at a concentration ranging from about 1 nM to 100 nM.

In any embodiment described herein, the subject's keratopathy may be a corneal dystrophy. More particularly, in any embodiment of the instant method, the keratopathy may be selected from the group consisting of: bacterial infections, viral infections, fungal infections, protozoan infections, archaeal infections, genetic disorders, brittle cornea syndrome (BCS), corneal endothelial-mesenchymal transition (EnMT), corneal epithelial-mesenchymal transition (EMT), corneal fibrosis, Cogan syndrome, corneal ulcer, epithelial basement membrane dystrophy (EBMD), Fleck corneal dystrophy, Fuchs' dystrophy, gelatinous droplike corneal dystrophy, granular corneal dystrophy type I, granular corneal dystrophy type II, interstitial keratitis, iridocorneal endothelial syndrome (ICE), keratoconjunctivitis sicca, keratoconus, keratomalacia, lattice dystrophy type I, lattice dystrophy type II, Lisch corneal dystrophy, macular corneal dystrophy, Meesmann corneal dystrophy, bullous keratopathy, aniridia, peripheral ulcerative keratitis, phlyctenular keratoconjunctivitis, posterior polymorphous corneal dystrophy, pterygium, Reis-Búckler corneal dystrophy, Schnyder crystalline corneal dystrophy, Stevens-Johnson syndrome, superficial punctate keratitis, neurotrophic keratitis, herpetic keratitis, Thiel-Behnke corneal dystrophy, scarring, limbal stem cell deficiency, and any combination thereof. In preferred embodiments of the instant method, the keratopathy is selected from: limbal stem cell deficiency (LSCD), epithelial-mesenchymal transition; endothelial-mesenchymal transition; corneal fibrosis; corneal scarring, including post-transplant corneal scarring; or any combination thereof. In a still preferred embodiment of the instant method, the keratopathy is Fuchs' dystrophy.

Also described herein is a method of increasing cell yield of mammalian cornea epithelium and/or endothelium cells grown ex vivo prior to or post cornea transplant, comprising treating a mammalian donor cornea or isolated corneal cells with WNT signaling modulator. In preferred embodiments, the mammalian donor cornea is a human cornea. In other embodiments the isolated corneal cells are limbal cells or corneal endothelial cells. In some embodiments, the WNT signaling modulator may be an engineered WNT signaling modulator. In some embodiments, the WNT signaling modulator may be an engineered WNT agonist.

In a preferred embodiment of the method of increasing cell yield, the engineered WNT agonist is selected from: (i) WNT3a; (ii) a WNT mimetic; (iii) an R-spondin mimetic, or (iv) a WNT mimetic is fused to a mutant Rspondin mimetic, referred to as a superSWAP or superagonist. In a still preferred embodiment, the WNT mimetic is a SWAP™ compound, the R-spondin mimetic is a SWEETS™ compound, or a SuperSWAP or superagonist.

In some embodiments, the disclosure provides methods, wherein the WNT signaling modulator is any of:
a. WNT3a or any homologue, mutant, fragment, mimic of WNT3a;
b. G211-18R5;
c. R2M3-26:
d. 1SH1-03;
e. hp1SH1-03
f. 17SB9-03,
g. any of the above alone or in combination with one or more Rspondin, or another growth factor such as a fibroblast growth factor (FGF);
h. G211-18R5-Rspo2RA or G211-R2H1-Rspo2RA or other SuperSWAPs or Superagonists:
i. or a combination thereof of any of the aforementioned.

In certain embodiments, the engineered WNT agonist comprises a sequence having at least 90%, at least 95%, at least 98%, at least 99%, or 100% identity to a sequence set forth in any of SEQ ID NOs:1-16. In particular embodiments, the engineered WNT agonist comprises one or more, e.g., two, sequence having at least 90%, at least 95%, at least 98%, at least 99%, or 100% identity to a sequence set forth in SEQ ID NO:1, and one or more, e.g., two, sequence having at least 90%, at least 95%, at least 98%, at least 99%, or 100% identity to a sequence set forth in SEQ ID NO:2. In particular embodiments, the engineered WNT agonist comprises one or more, e.g., two, sequence having at least 90%, at least 95%, at least 98%, at least 99%, or 100% identity to a sequence set forth in SEQ ID NO:3, and one or more, e.g., two, sequence having at least 90%, at least 95%, at least 98%, at least 99%, or 100% identity to a sequence set forth in SEQ ID NO:4. In particular embodiments, the engineered WNT agonist comprises one or more, e.g., two, sequence having at least 90%, at least 95%, at least 98%, at least 99%, or 100% identity to a sequence set forth in SEQ ID NO:5, and one or more, e.g., two, sequence having at least 90%, at least 95%, at least 98%, at least 99%, or 100% identity to a sequence set forth in SEQ ID NO:6. In particular embodiments, the engineered WNT agonist comprises two sequences having at least 90%, at least 95%, at least 98%, at least 99%, or 100% identity to a sequence set forth in SEQ ID NO:1, and two sequences having at least 90%, at least 95%, at least 98%, at least 99%, or 100% identity to a sequence set forth in SEQ ID NO:2. In particular embodiments, the engineered WNT agonist comprises two sequences having at least 90%, at least 95%, at least 98%, at least 99%, or 100% identity to a sequence set forth in SEQ ID NO:3, and two sequences having at least 90%, at least 95%, at least 98%, at least 99%, or 100% identity to a sequence set forth in SEQ ID NO:4. In particular embodiments, the engineered WNT agonist comprises two sequences having at least 90%, at least 95%, at least 98%, at least 99%, or 100% identity to a sequence set forth in SEQ ID NO:5, and two sequences having at least 90%, at least 95%, at least 98%, at least 99%, or 100% identity to a sequence set forth in SEQ ID NO:6.

In some embodiments, the engineered WNT agonist comprises two sequences having at least 90%, at least 95%, at least 98%, at least 99%, or 100% identity to a sequence set forth in SEQ ID NO:7, and two sequences having at least 90%, at least 95%, at least 98%, at least 99%, or 100% identity to a sequence set forth in SEQ ID NO:8.

In particular embodiments, the engineered WNT agonist comprises one or more, e.g., two, sequence having at least 90%, at least 95%, at least 98%, at least 99%, or 100% identity to a sequence set forth in SEQ ID NO:9, and one or more, e.g., two, sequence having at least 90%, at least 95%, at least 98%, at least 99%, or 100% identity to a sequence set forth in SEQ ID NO:10. In particular embodiments, the engineered WNT agonist comprises one or more, e.g., two, sequence having at least 90%, at least 95%, at least 98%, at least 99%, or 100% identity to a sequence set forth in SEQ ID NO:11, and one or more, e.g., two, sequence having at least 90%, at least 95%, at least 98%, at least 99%, or 100% identity to a sequence set forth in SEQ ID NO:12. In particular embodiments, the engineered WNT agonist comprises one or more, e.g., two, sequence having at least 90%, at least 95%, at least 98%, at least 99%, or 100% identity to a sequence set forth in SEQ ID NO:13, and one or more, e.g., two, sequence having at least 90%, at least 95%, at least 98%, at least 99%, or 100% identity to a sequence set forth in SEQ ID NO:14. In particular embodiments, the engineered WNT agonist comprises one or more, e.g., two, sequence having at least 90%, at least 95%, at least 98%, at least 99%, or 100% identity to a sequence set forth in SEQ ID NO:15, and one or more, e.g., two, sequence having at least 90%, at least 95%, at least 98%, at least 99%, or 100% identity to a sequence set forth in SEQ ID NO:16.

In any embodiment of the method of increasing cell yield, the WNT signaling modulator may be administered or co-administered with R-spondin, or any R-spondin homologue, mutant, fragment, mimic, or any combination thereof.

In preferred embodiments, the WNT signaling modulator is administered to the subject in a therapeutically effective dose. In any embodiment described herein, the WNT signaling modulator may be suspended in aqueous solution. The WNT signaling modulator may be suspended in aqueous solution at a concentration of about 0.01 pM, 0.1 pM, 1 pM, 0.01 nM, 0.1 nM, 1 nM, 10 nM, 100 nM, 1 μM, 10 μM, 100 μM, 1 mM, 10 mM, 100 mM, up to about 1 M. In preferred embodiments, the WNT signaling modulator is suspended in aqueous solution at a concentration ranging from about 0.1 nM to 1 μM. In still preferred embodiments, the WNT signaling modulator is suspended in aqueous solution at a concentration ranging from about 1 nM to 100 nM.

In preferred embodiments of the method of increasing cell yield, the WNT signaling modulator is administered to the mammalian donor cornea by bathing the mammalian donor cornea in vitro in an aqueous bath solution containing a dose of WNT signaling modulator. In other embodiments, the WNT signaling modulator is applied to limbal stem cells or corneal endothelial cells prior or post transplantation.

In preferred embodiments, the engineered WNT agonist and engineered WNT antagonist comprise binding compositions that bind to one or more Fzd receptors and binding compositions that bind to one or more LRP receptors. In still preferred embodiments of the method of increasing cell yield, the binding compositions of the engineered WNT agonist are selected from the group consisting of a Fzd binding composition, a Lrp5 binding composition, a Lrp6 binding composition, and a LRP5/6 binding composition.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts micrographs of fluorescent stained donor corneal endothelial tissue in healthy “normal” eye donors that have been stained for Fzd receptors, Fzd 1, 2, 4, 5, 7, 8, 9, and 10, using in situ hybridization, rendered here in green. Since Fzds1, 2, and 7 were the most prominently expressed, Fzds 1, 2, 7 expression in FECD patients has been the focus.

FIG. 1B depicts micrographs in additional healthy “normal” patients and in patients diagnosed with Fuchs' dystrophy, a keratopathic disorder. WNT and R-spondin receptors Lrp5/6; Fzd1/2/7; and ZNRF3, an E3 ubiquitin ligase, were stained using in situ hybridization, rendered here in green. The corneal endothelium marker Na+K+ ATPase was stained using immunofluorescence, rendered here in red. DNA is stained with 4′,6-diamidino-2-phenylindole (DAPI), rendered here in grey-white.

FIG. 2A depicts a graph of #Ki67+ (proliferation marker) cells per well in human corneal endothelial cells treated with FGF1, R-spondin-1, the WNT-mimetic compound R2M3-26, or the WNT superagonist or SuperSWAP G211-18R5-Rspo2RA. The results indicate that WNT mimetic and WNT superagonist or superSWAP increase proliferation of human corneal endothelial cells. * p≤0.05. ** p≤0.01. *** p≤0.001. Statistical analysis was performed using an unpaired two-tailed t test.

FIG. 2B depicts a graph of % Ki67+ cells in human corneal endothelial cells treated with FZD1/2/5/7/8+LRP6-binding WNT mimetic R2M3-26 or FZD1/2/7+LRP6-binding WNT mimetic 1RC07-26. The results indicate that WNT mimetics binding to FZD1/2/5/7/8 and LRP6 increase proliferation of human corneal endothelial cells. * p≤0.05. ** p≤0.01. Statistical analysis was performed using an unpaired two-tailed t test.

FIG. 2C depicts a graph of % Ki67+ cells in human corneal endothelial cells treated with the WNT superagonists or superSWAPs, G211-18R5-Rspo2RA or G211-R2H1-Rspo2RA. The results indicate that WNT superagonist or superSWAP binding to FZD1,2,7 and LRP6 is as effective as WNT superagonist or superSWAP G211-18R5-Rspo2RA (which binds to FZD1/2/5/7/8+LRP6-binding) in increasing proliferation of human corneal endothelial cells. * p≤0.05. ** p≤0.01. Statistical analysis was performed using an unpaired two-tailed t test.

FIG. 2D depicts fluorescence micrographs of untreated primary human corneal endothelial cells and primary human corneal endothelial cells treated with WNT mimetic G211-18R5+R-spondin-1. The images indicate that WNT agonists increase proliferation of cultured human corneal endothelial cells.

FIG. 2E depicts fluorescent micrographs of cornea from a human organ donor with Fuchs' dystrophy, both untreated and treated with WNT mimetic G211-18R5+R-spondin-1. The images indicate that WNT agonists increase proliferation of human corneal endothelial cells in cornea organ culture.

FIG. 3A depicts the change in Axin2 RNA transcript levels in corneas from mice injected intracamerally with anti-GFP isotype control antibody or wnt mimetic R2M3-26 (61 pmol each).

FIG. 3B depicts micrographs of fluorescent stained corneal endothelium from mice injected intracamerally with anti-GFP or SWAP, R2M3-26 (61 pmol each). WNT target gene Axin2 was stained using in situ hybridization, rendered here in green. DNA is stained with 4′,6-diamidino-2-phenylindole (DAPI), rendered here in blue.

FIG. 3C depicts the change in Axin2 RNA transcript levels in corneas from mice injected intracamerally (IC) or intravitreally (IVT) with anti-GFP or wnt superagonist or superSWAP, G211-18R5-Rspo2RA (2.6 mg each).

FIG. 3D depicts micrographs of fluorescent stained corneal endothelium from mice injected intracamerally with anti-GFP or G211-18R5-Rspo2RA (7 pmol each). WNT target gene Axin2 was stained using in situ hybridization, rendered here in green. DNA is stained with 4′,6-diamidino-2-phenylindole (DAPI), rendered here in blue.

FIGS. 3E-3F depict central corneal thickness measurements (FIG. 3E) and endothelial lesion size measurements (FIG. 3F) from rabbits 3 days after resection of the central 10 mm of the corneal endothelium. Rabbits were injected intracamerally on day 0 with anti-GFP or R2M3-26.

FIGS. 3G-3I, from a separate study, depict central corneal thickness measurements (FIG. 3H), endothelial lesion size (FIG. 3G) and corneal opacity (FIG. 3I) measurements from rabbits 3 days after resection of central 10 mm of the corneal endothelium. Rabbits were injected intracamerally on day 0 with anti-GFP or wnt superagonist or superSWAP, G211-18R5-Rspo2RA. The data in FIGS. 3G-3I show a significant improvement in the corneal opacity score with the high dose of wnt superagonist or superSWAP, while there appears to be a trend of decrease in corneal thickness in FIG. 3H.

FIGS. 4A-4C illustrate that the wnt superagonist or superSWAP, G211-18R5-Rspo2RA, reduces corneal thickness (FIG. 4A), improves clarity (FIG. 4B) and shows pharmacodynamic response (PD) (FIG. 4C) in a corneal cryoinjury mouse model. Relative to Anti-GFP isotype control, 5 μg of intravitreally (IVT) injected wnt superagonist or superSWAP, G211-18R5-Rspo2RA showed a significant reduction in central corneal thickness (compilation of 6 independent studies), as measured by optical coherence tomography (OCT) (FIG. 4A) and an improvement in corneal clarity in response to the cryoinjury (representative images from one such study) as measured by brightfield imaging of the anterior chamber of the eye (FIG. 4B). Wnt superagonist or superSWAP showed significant induction of Axin2 mRNA (Wnt target gene) on day 4 in corneal homogenates from one such study where G211-18R5-Rspo2RA or Anti-GFP antibody were administered intravitreally in the mice after cryoinjury (FIG. 4C).

FIGS. 5A-5B show that 1SH1-03 reduces % corneal thickness and improves corneal clarity in a corneal cryoinjury mouse model. FIG. 5A is a graph showing that relative to anti-GFP isotype control and 5 μg of 1SH1-03, 25 μg of intravitreally (IVT) injected 1SH1-03 showed a significant reduction in central corneal thickness (compilation of 2 independent studies) as measured by optical coherence tomography (OCT). At the 2 day timepoint, the lines from top to bottom correlate to: GFP, 1SH1-03 5 ug, and 1SH1-03 25 ug, respectively. FIG. 5B shows brightfield images showing that 1SH1-03 showed an improvement in corneal clarity in response to the cryoinjury as measured by brightfield imaging of the anterior chamber of the eye (representative images from one such study).

FIG. 6 are images showing that the Wnt mimetic, hp1SH1-03, shows a reduction in corneal endothelial lesion size in mice after corneal cryoinjury. In eyes after cryoinjury, intravitreally (IVT) injected hp1SH1-03 showed a reduction in lesion size relative to Anti-GFP isotype control. The figure depicts a corneal flat mount from eyes from the above treatment groups, which has been subjected to immunofluorescence and stained with Ki67 (rendered in red) and Dapi (in blue) and imaged on confocal microscope. While there is a lesion in the central region in the Anti-GFP isotype treated group which is devoid of proliferating Ki67 cells, there appears to be no detectable lesion in the hp1SH1-03-treated eyes and the region is marked by proliferating corneal endothelial cells.

FIGS. 7A-7B show that hp1SH1-03 induces robust proliferation in injured and uninjured human corneal endothelial cells (HCEC). Whole human corneas obtained from eye banks were cut into quarters, and treated with 5 nM of hp1SH1-03 for 2 days. FIG. 7A shows corneal endothelial cells were detected by ZO-1, and the proliferation rate was measured by counting the proportion of CECs expressing a proliferation marker, Ki67. FIG. 7B is the quantitative analysis, and reveals that hp1SH1-03 dramatically promotes HCEC proliferation, and this proliferation is more pronounced in the wound area; ˜6 times higher in the edge and ˜3 times higher in the mid-zone compared to anti-GFP treated eyes.

FIGS. 8A-8B show that the Wnt superagonist or superSWAP G211-18R5Rspo2RA shows increased PD response in the UV-A damage-induced FECD rodent model. FIG. 8A depicts the change in Axin2 RNA transcript levels in corneas from mice injected intravitreally (IVT) with anti-GFP or G211-18R5-Rspo2RA (2.6 ug each) in naïve mice or mice whose right eye was treated with 500 J/cm2 of UV-A light. FIG. 8B depicts central corneal thickness (CCT) in UV-A treated mice described in FIG. 8A and subjected to optical coherence tomography for the measurement of CCT as a measure of corneal swelling. There is a trend of decreased CCT in response to Wnt superagonist or superSWAP G211-18R5-Rspo2RA on day 2 post treatment.

FIGS. 9A-9B show expression levels of various markers. FIG. 9A depicts fluorescence micrographs of human cornea samples stained for various WNT receptors, cornea and limbal zonal markers, and DNA. Images are of cornea and limbal epithelium. FIG. 9B is a table indicating the expression levels of various WNT and R-spondin receptors in cornea epithelium and limbal epithelium.

FIGS. 10A-10B show expression of Axin2 and p63. FIG. 10A depicts the change in Axin2 RNA transcript levels in rabbit limbal epithelial organoids after 1 week of treatment with 5 nM WNT mimetic R2M3-26 or 1 μg/mL R-spondin-1. * p≤0.05. *** p≤0.001. Statistical analysis was performed using an unpaired two-tailed t test. FIG. 10B depicts the change in p63 RNA transcript levels in rabbit limbal epithelial organoids after 1 week of treatment with 5 nM WNT mimetic R2M3-26 or 1 μg/mL R-spondin-1. * p≤0.05. Statistical analysis was performed using an unpaired two-tailed t test.

FIGS. 11A-11C depict data indicating increased cell growth and proliferation in human corneal epithelium. FIG. 11A depicts cell growth data in cells treated for two days with WNT mimetic G211-18R5. Cell numbers were measured using CellTiter-Glo® assay. FIG. 11B depicts Ki67+ cell proliferation data after treatment with WNT mimetic R2M3-26. Proliferation was measured analyzing immunofluorescence micrographs. FIG. 11C depicts cell growth data in cells treated for two days with R-spondin-1. In each of Figs., statistical analysis was performed using an unpaired two-tailed t test.

FIGS. 12A-12B depict micrographs of an in vitro wound healing assay. Human corneal epithelial cells were plated around a physical barrier. The physical barrier was removed, leaving a gap. Cells were cultured were two days in a medium without (FIG. 12A) and with (FIG. 12B) WNT mimetic R2M3-26. Cells were then stained with DAPI and imaged, and the size of the gap was measured.

FIG. 13 shows that topical Fc-Rspo2 eye drops reduce corneal epithelial defects in corneal epithelial debridement mouse model. FIG. 13 depicts fluorescent and brightfield micrographs of central cornea epithelium from eyes of naïve mice, and mice 7 days after limbus-to-limbus debridement either treated with anti-GFP eyedrops (5 mg/mL, 3 μL, 4× a day) or Fc-Rspondin-2 eyedrops (5 mg/mL, 3 μL, 4× a day). WNT target gene Axin2 was stained using in situ hybridization, rendered here in red. DNA is stained with 4′,6-diamidino-2-phenylindole (DAPI), rendered here in blue. Progenitor marker p63 was stained using immunofluorescence, rendered here in green. Hematoxylin and eosin (H&E) staining is also depicted. The data illustrate that R-spondin eyedrops activate Wnt signaling, expand progenitor cells, thicken cornea epithelium and reduce conjunctivalization.

FIG. 14 depicts fluorescent micrographs of cornea from a human organ donor staining for THBD (Thrombomodulin/CD141/BDCA-3, a high affinity thrombin receptor present on endothelial cell membrane) showing specific staining in the membrane in the central cornea and the limbus but not in the conjunctiva), rendered here in green. DNA is stained with 4′,6-diamidino-2-phenylindole (DAPI), rendered here in blue. The data in FIG. 14 suggests that THBD can be employed for generating a limbal-targeted Rspo mimetic/SWEETs™ molecule for corneal regeneration.

FIG. 15A depicts a schematic representation of murine conjunctival and cornea epithelial organoids derived from primary tissue. Expanded cultures maintain markers such as keratin 12 (Ck12) or keratin 19 (Ck19).

FIG. 15B depicts the change in Axin2 mRNA transcript levels in conjunctival or cornea epithelium organoids treated with different WNT mimetics. R2M3-26 is a FZD12578 targeting WNT mimetic. 17SB9 is a FZD8 biased WNT mimetic. Data is normalized to untreated control per tissue. The data in FIG. 15B suggests that 17SB9-03-Rspo-mimetic can be for employed for generating a corneal epithelial-targeted Fzd8-Rspo mimetic/SWEETs™ molecule for corneal regeneration.

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

### I. Definitions

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

The terms “administering” or “introducing” or “providing”, as used herein, refer to delivery of a composition to a cell, to cells, tissues and/or organs of a subject, or to a subject. Such administering or introducing may take place in vivo, in vitro or ex vivo.

As used herein, the term “antibody” means an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Thus, it is used in the broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including, but not limited to, scFv, Fab, and Fab2, so long as they exhibit the desired biological activity.

“Antibody fragments” comprise a portion of an intact antibody, for example, the antigen-binding or variable region of the intact antibody. Examples of antibody fragments include Fab, Fab′, F(ab′)2, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

The term “antigen” refers to a molecule or a portion of a molecule capable of being bound by a selective binding agent, such as an antibody, and additionally capable of being used in an animal to produce antibodies capable of binding to an epitope of that antigen. In certain embodiments, a binding agent (e.g., a WNT surrogate molecule or binding region thereof, or a WNT antagonist) is said to specifically bind an antigen when it preferentially recognizes its target antigen in a complex mixture of proteins and/or macromolecules.

The term “antigen-binding fragment” as used herein refers to a polypeptide fragment that contains at least one complementarity-determining region (CDR) of an immunoglobulin heavy and/or light chain, or of a VHH/sdAb (single domain antibody) or Nanobody® (Nab), that binds to the antigen of interest, in particular to one or more Fzd receptors, or to LRP5 and/or LRP6. In this regard, an antigen-binding fragment of the herein described antibodies may comprise 1, 2, 3, 4, 5, or all 6 CDRs of a VH and VL from antibodies that bind one or more Fzd receptors or LRP5 and/or LRP6.

As used herein, the terms “biological activity” and “biologically active” refer to the activity attributed to a particular biological element in a cell. For example, the “biological activity” of a WNT agonist, or fragment or variant thereof refers to the ability to mimic or enhance WNT signals. As another example, the biological activity of a polypeptide or functional fragment or variant thereof refers to the ability of the polypeptide or functional fragment or variant thereof to carry out its native functions of, e.g., binding, enzymatic activity, etc. As a third example, the biological activity of a gene regulatory element, e.g., promoter, enhancer, Kozak sequence, and the like, refers to the ability of the regulatory element or functional fragment or variant thereof to regulate, i.e., promote, enhance, or activate the translation of, respectively, the expression of the gene to which it is operably linked.

The term “bifunctional antibody,” as used herein, refers to an antibody that comprises a first arm having a specificity for one antigenic site and a second arm having a specificity for a different antigenic site, i.e., the bifunctional antibodies have a dual specificity.

“Bispecific antibody” refers to a full-length antibody that is generated by quadroma technology (see Milstein et al., Nature, 305(5934): 537-540 (1983)), by chemical conjugation of two different monoclonal antibodies (see Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences) and is monovalent for each antigen to which it binds.

By “comprising,” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g., poly-adenylation sequence, enhancer elements, other genes, linker domains, etc.

By “consisting essentially of,” it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g., linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g., 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of,” it is meant the exclusion from the composition, method, or kit of any element, step, or ingredient not specified in the claim. For example, a polypeptide or polypeptide domain “consisting of” a recited sequence contains only the recited sequence.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

As used herein, “cornea” and “corneal” refers to the optically clear layered tissue which covers the iris, pupil, and anterior chamber of the eye. The “corneal limbus” or “limbus” refers to the more-or-less-circular border where the cornea meets the sclera of the eye. It should be readily understood to persons having ordinary skill in the art that the border between cornea tissue and limbus tissue is imprecise, and that reference to the “cornea” should be understood to encompass an approximate margin. The cornea comprises five cell layers: (1) epithelium (the external-most layer, a smooth optical surface and a barrier to chemicals, water, and microbes); (2) Bowman's layer (helps maintain the precise convex shape); (3) stroma (the main mechanical structure and refractive medium of the cornea); (4) Descemet's membrane (the resting layer for the endothelium); and (5) endothelium (maintains clarity by removing water from the stroma).

As used herein “Limbal cell deficiency” or “Limbal stem cell deficiency” is a genetic, acquired or idiopathic deficiency or complete loss of the stem cells in the limbus that are vital for re-population of the corneal epithelium. Loss or deficiency of Limbal stem cells leaves the corneal epithelium unable to repair or renew, leading to corneal conjunctivalization, neovascularization, corneal scarring, and chronic inflammation.

As used herein, “corneal dystrophy” refers generally to a disorder characterized by abnormal discoloration, clouding, and/or opacity of any layer or combination of layers of the cornea. Corneal dystrophies may be heritable or non-heritable. They may be caused by abnormal cell growth and/or by deposition and accumulation of collagens, lipids, cholesterols, and/or other extraneous material.

“Fuchs' dystrophy” (also called Fuchs' endothelial cell dystrophy (FECD), Fuchs' corneal dystrophy (FCD)) is a class of degenerative diseases of the cornea endothelium monolayer, characterized by a decline in endothelial cell density, corneal guttata, and an accumulation of corneal edema, leading to opacity of the cornea and a decline in vision.

As used herein, the term “engineered” indicates that a molecule, e.g., a Wnt signaling modulator, is non-naturally occurring, e.g., is the result of genetic engineering.

An “expression vector” is a vector, e.g., plasmid, minicircle, viral vector, liposome, and the like as discussed herein or as known in the art, comprising a region which encodes a gene product of interest, and is used for effecting the expression of the gene product in an intended target cell. An expression vector also comprises control elements, e.g., promoters, enhancers, untranslated regions (UTRs), miRNA targeting sequences, etc., operatively linked to the encoding region to facilitate expression of the gene product in the target. The combination of control elements and a gene or genes to which they are operably linked for expression is sometimes referred to as an “expression cassette,” a large number of which are known and available in the art or can be readily constructed from components that are available in the art.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

As used herein, the term “isolated” indicates the molecule, e.g., a naturally-occurring molecule, is not in its native environment or has been separated from one or more component present in its native environment or from one or more component present during manufacturing of the molecule.

As used herein, the terms “keratopathy” and “keratopathic” refer to disease or disorder of the eye, in particular of the cornea and/or limbus of the eye. Keratopathies include, but are not necessarily limited to, bacterial infections, viral infections (e.g., herpes simplex keratitis and herpes zoster ophthalmicus (i.e., shingles)), fungal infections, protozoan infections, archaeal infections, genetic disorders, brittle cornea syndrome (BCS), corneal endothelial-mesenchymal transition (EnMT), corneal epithelial-mesenchymal transition (EMT), corneal fibrosis, Cogan syndrome, corneal ulcer, epithelial basement membrane dystrophy (EBMD, ABMD, Map Dot), Fleck corneal dystrophy, Fuchs' dystrophy (also called Fuchs' corneal endothelial dystrophy (FCED) or Fuchs' endothelial dystrophy (FED)), gelatinous droplike corneal dystrophy, granular corneal dystrophy type I, granular corneal dystrophy type II, interstitial keratitis, iridocorneal endothelial syndrome (ICE), keratoconjunctivitis sicca, keratoconus, keratomalacia, lattice dystrophy type I, lattice dystrophy type II, Lisch corneal dystrophy, macular corneal dystrophy, Meesmann corneal dystrophy, bullous keratopathy, aniridia, peripheral ulcerative keratitis, phlyctenular keratoconjunctivitis, posterior polymorphous corneal dystrophy, pterygium, Reis-Bückler corneal dystrophy, Schnyder crystalline corneal dystrophy, Stevens-Johnson syndrome, superficial punctate keratitis, Thiel-Behnke corneal dystrophy, neurotrophic keratitis, herpetic keratitis, scarring, and limbal stem cell deficiency.

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), Nanobodies®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including WNT surrogate molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

The term “native” or “wild-type” as used herein refers to a nucleotide sequence, e.g., gene, or gene product, e.g., RNA or protein, that is present in a wild-type cell, tissue, organ, or organism. The term “variant” as used herein refers to a mutant of a reference polynucleotide or polypeptide sequence, for example a native polynucleotide or polypeptide sequence, i.e., having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g., a native polynucleotide or polypeptide sequence. For example, a variant may be a polynucleotide having a sequence identity of 50% or more, 60% or more, or 70% or more with a full-length native polynucleotide sequence, e.g., an identity of 75% or 80% or more, such as 85%, 90%, or 95% or more, for example, 98% or 99% identity with the full length native polynucleotide sequence. As another example, a variant may be a polypeptide having a sequence identity of 70% or more with a full-length native polypeptide sequence, e.g., an identity of 75% or 80% or more, such as 85%, 90%, or 95% or more, for example, 98% or 99% identity with the full-length native polypeptide sequence. Variants may also include variant fragments of a reference, e.g., native, sequence sharing a sequence identity of 70% or more with a fragment of the reference, e.g., native, sequence, e.g., an identity of 75% or 80% or more, such as 85%, 90%, or 95% or more, for example, 98% or 99% identity with the native sequence.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double-stranded and single-stranded molecules. Unless otherwise specified or required, any embodiment of described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. As used herein, the terms “identity” and “identical” refer, with respect to a polypeptide or polynucleotide sequence, to the percentage of exact matching residues in an alignment of that “query” sequence to a “subject” sequence, such as an alignment generated by the BLAST algorithm. Identity is calculated, unless specified otherwise, across the full length of the subject sequence. Thus, a query sequence “shares at least x % identity to” a subject sequence if, when the query sequence is aligned to the subject sequence, at least x % (rounded down) of the residues in the subject sequence are aligned as an exact match to a corresponding residue in the query sequence. Where the subject sequence has variable positions (e.g., residues denoted X), an alignment to any residue in the query sequence is counted as a match. Sequence alignments are performed using the NCBI Blast service (BLAST+ version 2.12.0) unless otherwise indicated. To determine sequence identity, sequences can be aligned using the methods and computer programs, including BLAST, available over the worldwide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in METHODS IN ENZYMOLOGY, VOL. 266: COMPUTER METHODS FOR MACROMOLECULAR SEQUENCE ANALYSIS (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith and Waterman (Adv. Appl. Math. 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences input to be compared. Preferably, the sequence identity is determined using the default parameters determined by the program. This program is available also from Genetics Computing Group (GCG) package, from Madison, Wis., USA. Another program of interest is the FastDB algorithm. FastDB is described in CURRENT METHODS IN SEQUENCE COMPARISON AND ANALYSIS, MACROMOLECULE SEQUENCING AND SYNTHESIS, SELECTED METHODS AND APPLICATIONS, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the disclosed nucleic acid constructs or vectors are enhancer sequences that may or may not be contiguous with the promoter sequence. Enhancer sequences influence promoter-dependent gene expression and may be located in the 5′ or 3′ regions of the native gene.

“Recombinant,” as applied to a polynucleotide means that the polynucleotide is the product of various combinations of cloning, restriction or ligation steps, and other procedures that result in a construct that is distinct from a polynucleotide found in nature.

As used herein, “SWAP™” (Surrozen WNT-signal activating proteins) refers to WNT mimetic compounds comprising engineered bi-specific full-length immunoglobulin-G (IgG) antibodies that, like WNT proteins, directly activate the canonical WNT-signaling pathway in a target tissue, e.g., cornea tissues.

As used herein, “SWEETS™” (Surrozen WNT-signal enhancer engineered for tissue specificity) refers to antibody-based R-spondin mimetic compounds.

The terms “treatment”, “treating” and the like are used herein to generally mean obtaining a desired pharmacologic and/or physiologic effect. The effect may be prophylactic in terms of completely or partially preventing a disease or symptom thereof, e.g., reducing the likelihood that the disease or symptom thereof occurs in the subject, and/or may be therapeutic in terms of a partial or complete cure for a disease and/or adverse effect attributable to the disease.

“Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The practice of the present disclosure will employ, unless otherwise indicated, conventional techniques of cell biology, molecular biology techniques, microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the disclosure are described below with reference to example applications for illustration. It should be understood that numerous specific details, relationships, and methods are set forth to provide a full understanding of the disclosure. One having ordinary skill in the relevant art, however, will readily recognize that the disclosure can be practiced without one or more of the specific details or with other methods. The present disclosure is not limited by the illustrated ordering of acts or events, as some acts may occur in different orders and/or concurrently with other acts or events. Furthermore, not all illustrated acts or events are required to implement a methodology in accordance with the present disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Unless otherwise indicated, all terms used herein have the same meaning as they would to one skilled in the art and the practice of the present disclosure will employ, conventional techniques of microbiology and recombinant DNA technology, which are within the knowledge of those of skill of the art.

### II. General

The present disclosure provides methods of modulating WNT signals to treat cornea disorders, including but not limited to, scarring, and corneal epithelial and endothelial dystrophies. In particular, the present disclosure provides a WNT/0-catenin agonist and/or antagonist to stimulate corneal tissue regeneration and/or prevent corneal fibrosis.

WNT (“Wingless-related integration site” or “Wingless and Int-1” or “Wingless-Int”) ligands and their signals play key roles in the control of development, homeostasis and regeneration of many essential organs and tissues, including bone, liver, skin, stomach, intestine, kidney, central nervous system, mammary gland, taste bud, ovary, cochlea, lung, and many other tissues (reviewed, e.g., by Clevers, Loh, and Nusse, 2014; 346:1248012). Modulation of WNT signaling pathways has potential for treatment of degenerative diseases and tissue injuries.

One of the challenges for modulating WNT signaling as a therapeutic is the existence of multiple WNT ligands and WNT receptors, Frizzled 1-10 (Fzd1-10), with many tissues expressing multiple and overlapping Fzds. Canonical WNT signals also involve Low-density lipoprotein (LDL) receptor-related protein 5 (LRP5) or Low-density lipoprotein (LDL) receptor-related protein 6 (LRP6) as co-receptors, which are broadly expressed in various tissues, in addition to Fzds.

R-spondins 1-4 are a family of ligands that amplify WNT signals. Each of the R-spondins work through a receptor complex that contains Zinc and Ring Finger 3 (ZNRF3) or Ring Finger Protein 43 (RNF43) on one end and a Leucine-rich repeat-containing G-protein coupled receptor 4-6 (LGR4-6) on the other (reviewed, e.g., by Knight & Hankenson 2014, Matrix Biol.; 37: 157-161). R-spondins might also work through additional mechanisms of action. ZNRF3 and RNF43 are two membrane-bound E3 ligases specifically targeting WNT receptors (Fzd1-10 and LRP5 or LRP6) for degradation. Binding of an R-spondin to ZNRF3/RNF43 and LGR4-6 causes clearance or sequestration of the ternary complex, which removes E3 ligases from WNT receptors and stabilizes WNT receptors, resulting in enhanced WNT signals. Each R-spondin contains two Furin domains (1 and 2), with Furin domain 1 binding to ZNRF3/RNF43, and Furin domain 2 binding to LGR4-6. Fragments of R-spondins containing Furin domains 1 and 2 are sufficient for amplifying WNT signaling. While R-spondin effects depend on WNT signals, since both LGR4-6 and ZNRF3/RNF43 are widely expressed in various tissues, the effects of R-spondins are not tissue-specific.

In some embodiments, the WNT/β-catenin signaling antagonist or agonist can include binding agents or epitope binding domains that bind one or more Fzd receptors and inhibit or enhance WNT signaling. In certain embodiments, the agent or antibody specifically binds to the cysteine-rich domain (CRD) within the human frizzled receptor(s) to which it binds. Additionally, antagonistic binding agents containing epitope binding domains against LRP can also be used. In some embodiments, the WNT/β-catenin antagonist possesses binding agents or epitope binding domains that bind E3 ligases ZNRF3/RNF43 and one or more FZD receptors or one or more LRP co-receptors to promote the degradation of FZD or LRP receptors, and this molecule can also contain a binding domain that binds a cell type specific epitope for targeting. The E3 ligase agonist antibodies or fragments thereof can be single molecules or combined with other WNT antagonists, e.g., Fzd receptor antagonists, LRP receptor antagonists, etc.

As is well known in the art, an antibody is an immunoglobulin molecule capable of specific binding to a target such as a carbohydrate, polynucleotide, lipid, polypeptide, etc., through at least on epitope binding domain, located on the variable region of the immunoglobulin molecule. As used herein, the term encompasses not only intact polyclonal or monoclonal antibodies, but also fragments thereof containing epitope binding domains (e.g., dAb, Fab, Fab′, (F(ab′)₂, Fv, single chain (scFv), VHH (i.e., Nanobodies®) or single domain antibodies (sdAb), DVD-Igs, synthetic variants thereof, naturally occurring variants, fusion proteins comprising and epitope binding domain, humanized antibodies, chimeric antibodies, and any other modified configuration of the immunoglobulin molecule that comprises an antigen-binding site or fragment (epitope recognition site) of the required specificity. “Diabodies,” multivalent or multispecific fragments constructed by gene fusion (WO94/13804; P. Holliger et al., Proc. Natl. Acad. Sci. USA 90 6444-6448, 1993) are also a particular form of antibody contemplated herein. Minibodies comprising a scFv joined to a CH3 domain are also included herein (S. Hu et al., Cancer Res., 56, 3055-3061, 1996). See e.g., Ward, E. S. et al., Nature 341, 544-546 (1989); Bird et al., Science, 242, 423-426, 1988; Huston et al., Proc. Natl. Acad. Sci. USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996; C. Bever et al., Anal Bioanal Chem. 2016 September; 408(22); 5985-6002.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments of the present disclosure can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH:VL heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In certain embodiments, single chain Fv (scFV) antibodies are contemplated. For example, Kappa bodies (Ill et al., Prot. Eng. 10: 949-57 (1997)); minibodies (Martin et al., EMBO J 13: 5305-9 (1994)); diabodies (Holliger et al., Proc. Nat. Acad. Sci. 90: 6444-8 (1993)); or Janusins (Traunecker et al., EMBO J 10: 3655-59 (1991) and Traunecker et al., Int. J. Cancer Suppl. 7: 51-52 (1992)), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to one or more Fzd receptors through one binding domain and to a second molecule through a second binding domain. These antibodies may be produced through recombinant molecular biological techniques or may be physically conjugated together.

A single chain Fv (scFv) polypeptide is a covalently linked VH:VL heterodimer which is expressed from a gene fusion including VH- and VL-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See. e.g., U.S. Pat. Nos. 5,091,513 and 5,132,405, to Huston et al.; and U.S. Pat. No. 4,946,778, to Ladner et al.

In certain embodiments, an antibody as described herein is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen binding site: antigen binding sites are formed by the association of the first domain of one polypeptide within the multimer with the second domain of another polypeptide within the multimer (WO94/13804).

A dAb fragment of an antibody consists of a VH domain (Ward, E. S. et al., Nature 341, 544-546 (1989)).

Where bispecific antibodies are to be used, these may be conventional bispecific antibodies, which can be manufactured in a variety of ways (Holliger, P. & Winter G., Curr. Opin. Biotech. 4, 446-449 (1993)), e.g., prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied, and an antibody of appropriate specificity selected. Bispecific whole antibodies may be made by knobs-into-holes engineering (J. B. B. Ridgeway et al., Protein Eng., 9, 616-621 (1996)).

In certain embodiments, the antibodies described herein may be provided in the form of a UniBody®. A UniBody® is an IgG4 antibody with the hinge region removed (see GenMab Utrecht, The Netherlands; see also, e.g., US2009/0226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), Nanobodies®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including WNT surrogate molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

In certain embodiments, the antibodies of the present disclosure may take the form of a Nanobody®. Nanobody® technology was originally developed following the discovery and identification that camelidae (e.g., camels, alpacas, and llamas) possess fully functional antibodies that consist of heavy chains only and therefore lack light chains. These heavy-chain only antibodies contain a single variable domain (VHH) and two constant domains (CH2, CH3). The cloned and isolated single variable domains have full antigen binding capacity and are very stable. These single variable domains, with their unique structural and functional properties, form the basis of “Nanobodies®”. Nanobodies® are encoded by single genes and are efficiently produced in almost all prokaryotic and eukaryotic hosts, e.g., E. coli (see, e.g., U.S. Pat. No. 6,765,087), molds (for example Aspergillus or Trichoderma) and yeast (for example Saccharomyces, Kluyvermyces, Hansenula, or Pichia (see, e.g., U.S. Pat. No. 6,838,254). The production process is scalable and multi-kilogram quantities of Nanobodies® have been produced. Nanobodies® may be formulated as a ready-to-use solution having a long shelf life. The Nanoclone® method (see, e.g., WO 06/079372) is a proprietary method for generating Nanobodies® against a desired target, based on automated high-throughput selection of B-cells. Nanobodies® are single-domain antigen-binding fragments of camelid-specific heavy-chain only antibodies. Nanobodies®, also referred to as VHH antibodies, typically have a small size of around 15 kDa. See C. Bever et al., Anal Bioanal Chem. 2016 September; 408(22); 5985-6002.

Another antibody fragment contemplated is a dual-variable domain-immunoglobulin (DVD-Ig) is an engineered protein that combines the function and specificity of two monoclonal antibodies in one molecular entity. A DVD-Ig is designed as an IgG-like molecule, except that each light chain and heavy chain contains two variable domains in tandem through a short peptide linkage, instead of one variable domain in IgG. The fusion orientation of the two variable domains and the choice of linker sequence are critical to functional activity and efficient expression of the molecule. A DVD-Ig can be produced by conventional mammalian expression systems as a single species for manufacturing and purification. A DVD-Ig has the specificity of the parental antibodies, is stable in vivo, and exhibits IgG-like physicochemical and pharmacokinetic properties. DVD-Igs and methods for making them are described in Wu, C., et al., Nat Biotech, 25:1290-1297 (2007).

In certain embodiments, the antibodies or antigen-binding fragments thereof as disclosed herein are humanized. This refers to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio, A. F. et al., (1989) Proc Natl Acad Sci USA 86:4220-4224; Queen et al., PNAS (1988) 86:10029-10033; Riechmann et al., Nature (1988) 332:323-327). Illustrative methods for humanization of the anti-Fzd or LRP antibodies disclosed herein include the methods described in U.S. Pat. No. 7,462,697.

Another approach focuses not only on providing human-derived constant regions but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856; Riechmann, L., et al., (1988) Nature 332:323-327; Verhoeyen, M., et al., (1988) Science 239:1534-1536; Kettleborough, C. A., et al., (1991) Protein Engineering 4:773-3783; Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio/Technology 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992) J Immunol 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present disclosure may be chimeric antibodies. In this regard, a chimeric antibody is comprised of an antigen-binding fragment of an antibody operably linked or otherwise fused to a heterologous Fc portion of a different antibody. In certain embodiments, the heterologous Fc domain is of human origin. In other embodiments, the heterologous Fc domain may be from a different Ig class from the parent antibody, including IgA (including subclasses IgAQ1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

The structures and locations of immunoglobulin CDRs and variable domains may be determined by reference to Kabat, E. A. et al., SEQUENCES OF PROTEINS OF IMMUNOLOGICAL INTEREST, 4th Edition, US Department of Health and Human Services. 1987, and updates thereof, now available on the Internet (immuno.bme.nwu.edu).

In certain embodiments, the antagonist or agonist binding agent binds with a dissociation constant (K_{D}) of about 1 μM or less, about 100 nM or less, about 40 nM or less, about 20 nM or less, or about 10 nM or less. For example, in certain embodiments, a FZD binding agent or antibody described herein that binds to more than one FZD, binds to those FZDs with a K_{D} of about 100 nM or less, about 20 nM or less, or about 10 nM or less. In certain embodiments, the binding agent binds to one or more its target antigen with an EC50 of about 1 μM or less, about 100 nM or less, about 40 nM or less, about 20 nM or less, about 10 nM or less, or about 1 nM 20 or less.

The antibodies or other agents of the present disclosure can be assayed for specific binding by any method known in the art. The immunoassays which can be used include, but are not limited to, competitive and non-competitive assay systems using techniques such as biolayer interferometry (BLI) analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see. e.g., Ausubel et al, eds, 1994, CURRENT PROTOCOLS IN MOLECULAR BIOLOGY, Vol. 1, John Wiley & Sons, Inc., New York, which is incorporated by reference herein in its entirety).

For example, the specific binding of an antibody to a target antigen may be determined using ELISA. An ELISA assay comprises preparing antigen, coating wells of a 96 well microtiter plate with antigen, adding the antibody or other binding agent conjugated to a detectable compound such as an enzymatic substrate (e.g., horse-radish peroxidase or alkaline phosphatase) to the well, incubating for a period of time and detecting the presence of the antigen. In some embodiments, the antibody or agent is not conjugated to a detectable compound, but instead a second conjugated antibody that recognizes the first antibody or agent is added to the well. In some embodiments, instead of coating the well with the antigen, the antibody or agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs known in the art (see. e.g., Ausubel et al, eds, 1994, CURRENT PROTOCOLS IN MOLECULAR BIOLOGY, Vol. 1, John Wiley & Sons, Inc., New York at 11.2.1).

The binding affinity of an antibody or other agent to a target antigen and the off-rate of the antibody-antigen interaction can be determined by competitive binding assays. One example of a competitive binding assay is a radioimmunoassay comprising the incubation of labeled antigen (e.g., Fzd, LRP), or fragment or variant thereof, with the antibody of interest in the presence of increasing amounts of unlabeled antigen followed by the detection of the antibody bound to the labeled antigen. The affinity of the antibody and the binding off-rates can be determined from the data by scattered plot analysis. In some embodiments, BLI analysis is used to determine the binding on and off rates of antibodies or agents. BLI kinetic analysis comprises analyzing the binding and dissociation of antibodies from chips with immobilized antigens on their surface.

In certain embodiments, the WNT agonist is selected from those disclosed in PCT Publication No. WO 2019/126398, which is incorporated herein in its entirety. G211 (also known as YW211.31.57) is disclosed in U.S. Pat. No. 8,846,041.

In some embodiments, a WNT agonist comprises a sequence having at least 90% identity (e.g., 95%, 98%, 99% or 100% identity) to a sequence disclosed in any of the sequences depicted in Table 1. In some embodiments, a WNT agonist comprises two sequences, each having at least 90% identity (e.g., 95%, 98%, 99% or 100% identity) to a sequence disclosed in any of the sequences depicted in Table 1. In some embodiments, a WNT agonist comprises four sequences, each having at least 90% identity (e.g., 95%, 98%, 99% or 100% identity) to a sequence disclosed in any of the sequences depicted in Table 1, and in particular embodiments, two of the sequences are light chain sequences, and two of the sequences are heavy chain sequences. In some embodiments, a WNT agonist comprises two heavy chain sequences. In certain embodiments, the WNT agonist comprises a light chain sequence and/or a heavy chain sequence, each independently comprising at least one, at least two, or all three CDR sequences disclosed in any of SEQ ID NOs: 1-16, or variants thereof wherein the sequence comprises zero, less than two, less than three, less than four, less than five, less than six, less than seven, or less than eight amino acid modifications, e.g., substitutions, within the CDRs. G211-18R5, R2M3-26, 1RCO7-26, 1SH1-03 and hp1SH1-03 comprise two of the indicated heavy chains (HC) and two of the indicated light chains (LC) disclosed in Table 1. In certain embodiments, the WNT agonist comprises two light chain sequences and two heavy chain sequence, each independently comprising at least one, at least two, or all three CDR sequences disclosed in any of SEQ ID NOs: 1-16, or variants thereof. In certain embodiments, the Wnt agonist has an Ig format, and in certain embodiments, the Wnt agonist comprises two heavy chains bound to each other, and two light chains, each bound to a different one of the two heavy chains. Wnt superagonists or superSWAPs G211-18R5-Rspo2RA and G211-R2H1-Rspo2RA are also listed in Table 1; they each comprises two of the indicated heavy chains (HC) and two of the indicated light chains (LC) disclosed in Table 1. 17SB9-03 comprise two of the indicated heavy chains (HC) disclosed in Table 1, i.e., one HC of SEQ ID NO: 15 and one heavy chain of SEQ ID NO:16. In various embodiments, the molecules do not include the tag sequences.

Table 1: Sequences of SWAP™ and Wnt superagonists or superSWAP molecules; the variable chains are provided in plain text in the order indicated by the format; when present, the VHH is in bold (not italics); and the constant chain is in italics. The VL is in italics and underlined; the VH is in italics and bold. The constant region of the lambda light chain is underlined and in smaller font. The constant region of the kappa light chain is underlined, bold, and in smaller font. The CH1 is in italics. The Fc is in italics, bold, and in smaller font. The Rspo region is bold and in smaller font (not italics or underline). The linker is in smaller font. The bold, underlined and italicized AA and G represent substitutions of the wild-type LL and P amino acids at these positions with AA and G, respectively. Tag sequences are underlined with a dotted line.

G211-18R5_LC
Format: G211. VK-GS linker-18R5.VL-hCL (constant lambda light chain)
(SEQ ID NO: 1)
DIQMTQSPSSLSASVGDRVTITCRASQDVSTAVAWYQQKPGKAPKLLIYSASFLYSGVPSRF
SGSGSGTDFTLTISSLQPEDFATYYCQQSYTTPPTFGQGTKVEIKGGGGSGGGGSGGGGSDIE
LTQPPSVSVAPGQTARISCSGDNIGSFYVHWYQQKPGQAPVLVIYDKSNRPSGIPERFSGS
NSGNTATLTISGTQAEDEADYYCQSYANTLSLVFGGGTKLTVLGQPKAAPSVTLFPPSSEELQA
NKATLVCLISDFYPGAVTVAWKADSSPVKAGVETTTPSKQSNNKYAASSYLSLTPEQWKSHRSYSCQ
VTHEGSTVEKTVAPTECS
G211-18R5_HC
Format: G211. VH- GS linker-18R5. VH-hIgGI CHI-CH3 LALAPG
(constant heavy chain)
(SEQ ID NO: 2)
EVQLVESGGGLVQPGGSLRLSCAASGFTFTSYYISWVRQAPGKGLEWVAEISPYSGST
YYADSVKGRFTISADTSKNTAYLQMNSLRAEDTAVYYCALRARPPIRLHPRGSVMDY
WGQGTLVTVSSGGGGSGGGGSGGGGSEVQLVESGGGLVQPGGSLRLSCAASGFTFSHY
TLSWVRQAPGKGLEWVSVISGDGSYTYYADSVKGRFTISSDNSKNTLYLQMNSLRAE
DTAVYYCARNFIKYVFANWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNT
KVDKKVEPKSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALGAPIEKTISKAKGQP
REPQVYTLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
R2M3-26_LC
Format: VHH26-GS linker-R2M3.VL-hCL (constant lambda light chain)
(SEQ ID NO: 3)
DVQLVESGGGLVQAGGSLRLACAGSGRIFAIYDIAWYRHPPGNQRELVAMIRP
VVTEIDYADSVKGRFTISRNNAMKTVYLQMNNLKPEDTAVYYCNAKRPWGSRD
EYWGQGTQVTVSSGSGSGQAVVLQEPSLSVSPGGTVTLTCGLSSGSVSTNYYPSWYQQTP
GQAPRTLIYYTNTRSSDVPERFSGSIVGNKAALTITGAQPDDESVYFCLLYLGRGIWVFGGG
TKLTVLGQPKAAPSVTLFPPSSEELQANKATLVCLISDFYPGAVTVAWKADSSPVKAGVETTTPSKQS
NNKYAASSYLSLTPEQWKSHRSYSCQVTHEGSTVEKTVAPTECS
R2M3-26_HC
Format: R2M3. VH-hIgGI CHI-CH3 LALAPG (constant heavy chain)
(SEQ ID NO: 4)
EVQLVQSGAEVKKPGASVKVSCKASGYTFTSYGISWVRQAPGQGLEWMGWISAYNG
NTNYAQKLQGRVTMTTDTSTSTAYMELRSLRSDDTAVYYCASSKEKATYYYGMDVW
GQGTTVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHTCPPCPA
PEAAGGPSVFLFPPKPKDTLMISRTPEVTCVVVDVSHEDPEVKFNWYVDGVEVHNAKTKPREEQYNST
YRVVSVLTVLHQDWLNGKEYKCKVSNKALGAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHEALHNH
YTQKSLSLSPGK
1RC07-26_LC
Format: VHH26-GS linker-1RC07.VL-hCL (constant lambda light chain)
(SEQ ID NO: 5)
DVQLVESGGGLVQAGGSLRLACAGSGRIFAIYDIAWYRHPPGNQRELVAMIRP
VVTEIDYADSVKGRFTISRNNAMKTVYLQMNNLKPEDTAVYYCNAKRPWGSRD
EYWGQGTQVTVSSGSGSGSYVLTQPPSVSVSPGQTASITCSGDKVGHKYASWYQQKPGQ
SPVLVIYEDSQRPSGIPVRESGSNSGNTATLTISGTQAMDEADYYCQAWDSSTDVVFGGGT
KLTVLGQPKAAPSVTLFPPSSEELQANKATLVCLISDFYPGAVTVAWKADSSPVKAGVETTTPSKQSN
INKYAASSYLSLTPEQWKSHRSYSCQVTHEGSTVEKTVAPTECS
1RC07-26_HC
Format: 1RC07.VH-hIgGI CHI-CH3 LALAPG (constant heavy chain)
(SEQ ID NO: 6)
QVQLQQWGAGLLKPSETLSLTCAVSGASFSGHYWTWIRQPPGKGLEWIGEIDHTGS
TNYEPSLRSRVTISVDTSKNQFSLNLKSVTAADTAVYYCARGGQGGYDWGHYHGLD
VWGQGTTVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTHTCPP
CPAPEAAGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALGAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEAL
HNHYTQKSLSLSPGK
hp-1SH1-03_LC
Format: VHH03-GS linker-hp-1SH1 VL-hCK (constant kappa light chain)
(SEQ ID NO: 7)
EVQLVESGGGLVQPGGSLRLSCASSANIQSIETLGWYRQAPGKQRELIANMRG
GGYMKYADSLKGRFTMSTDNSKNTMYLQMNSLRAEDTAVYYCYVKLRDEDY
VYRGQGTQVTVSSGGGGSDIVMTQSPLSLPVTPGEPASISCRSSQSLLHSGGYTYLDWYL
QKPGQSPQLLIYLGSKRASGVPDRFSGSGSGTDFTLKISRVEAEDVGVYYCMQALQIPPTF
GQGTKVEIKRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
hp-1SH1-03_HC
Format: hp-1SH1. VH- hIgGI CHI-CH3 LALAPG (constant heavy chain)
(SEQ ID NO: 8)
EVQLQESGPGLVKPSQTLSLTCTVSGGSISSGGYYWSWIRQHPGKGLEWIGYIYYSGS
TYYNPSLKSRVTISVDTSKNQFSLKLSSVTAADTAVYYCARHAGFYGLADYFDYWGQ
GTLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALGAPIEKTISKAKGQPREPQVYTLPPSREEMTKNQVSLTCLVK
GFYPSDIAVEWESNGQPENNYKTTPVLDSDGSFFLYSKLTVDKSRWQQGNVFSCSVMHEALHNHYTQ
KSLSLSPGK
1SH1-03_LC
Format: VHH3-GS linker-1SH1 VL-hCK (constant kappa light chain)
(SEQ ID NO: 9)
DVQLVESGGGLVQPGGSLRLSCTSSANINSIETLGWYRQAPGKQRELIANMRGG
GYMKYAGSLKGRFTMSTESAKNTMYLQMNSLKPEDTAVYYCYVKLRDDDYV
YRGQGTQVTVSSGGSGSDVVMTQSPLSLPVTPGEPASISCRSSQSLLHSNGYNYLDWYLQ
KPGQSPQLLIYLGSKRASGVPDRFSGSGSGTDFTLKISRVEAEDVGVYYCMQALQIPPTFG
QGTKVEIKRTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
1SH1-03_HC
Format: 1SH1. VH- hIgGI CHI-CH3 LALAPG (constant heavy chain)
(SEQ ID NO: 10)
EVQLVESGPGLVKPSQTLSLTCTVSGGSISSGGYYWSWIRQHPGKGLEWIGYIYYSGS
TYYNPSLKSRVTISVDTSKNQFSLKLNSVTAADTAVYYCARHAGFYGLADYFDYWGQ
GTLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALGAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEALHNHYT
QKSLSLSPGK
G211-18R5-LC
Format: G211.VL-GS linker-18R5.VL-hCL (constant lambda light chain)
(SEQ ID NO: 11)
DIQMTQSPSSLSASVGDRVTITCRASQDVSTAVAWYQQKPGKAPKLLIYSASFLYSGVPSRF
SGSGSGTDFTLTISSLQPEDFATYYCQQSYTTPPTFGQGTKVEIKGGGGSGGGGGGGGSDIE
LTQPPSVSVAPGQTARISCSGDNIGSFYVHWYQQKPGQAPVLVIYDKSNRPSGIPERFSGS
NSGNTATLTISGTQAEDEADYYCQSYANTLSLVFGGGTKLTVLGQPKAAPSVTLFPPSSEELQA
NKATLVCLISDFYPGAVTVAWKADSSPVKAGVETTTPSKQSNNKYAASSYLSLTPEQWKSHRSYSCQ
VTHEGSTVEKTVAPTECS
G211-18R5-Rspo2RA_HC
Format: G211. VH-GS linker-18R5.VH-hIgGI CHI-CH3 LALAPG
(constant heavy chain)-GS linker-Rspo2RA
(SEQ ID NO: 12)
EVQLVESGGGLVQPGGSLRLSCAASGFTFTSYYISWVRQAPGKGLEWVAEISPYSGST
YYADSVKGRFTISADTSKNTAYLQMNSLRAEDTAVYYCALRARPPIRLHPRGSVMDY
WGQGTLVTVSSGGGGSGGGGSGGGGSEVQLVESGGGLVQPGGSLRLSCAASGFTFSHY
TLSWVRQAPGKGLEWVSVISGDGSYTYYADSVKGRFTISSDNSKNTLYLQMNSLRAE
DTAVYYCARNFIKYVFANWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNT
KVDKKVEPKSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALGAPIEKTISKAKGQP
REPQVYTLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGKGGGGSGSGGSGGGGSNPICKGCLSCSKDNGC
SRCQQKLFFFLRREGMRQYGECLHSCPSGYYGHRAPDMNRCARCRIENCDSCRSKDACTKCKV
GFYLHRGRCFDECPDGFAPLEETMECVE
G211-R2H1-LC
Format: G211. VL-GS linker-R2H1. VL-hCK (constant kappa light chain)
(SEQ ID NO: 13)
DIQMTQSPSSLSASVGDRVTITCRASQDVSTAVAWYQQKPGKAPKLLIYSASFLYSGVPSRF
SGSGSGTDFTLTISSLQPEDFATYYCQQSYTTPPTFGQGTKVEIKGGGGSGGGGSGGGGSDIQ
MTQSPSSLSASVGDRVTITCRASQSVSSAVAWYQQKPGKAPKLLIYSASSLYSGVPSRFSGSR
SGTDFTLTISSLQPEDFATYYCQQYWYGVAPITFGQGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGEC
G211-R2H1-Rspo2RA-HC
Format: G211.VH-GS linker-R2H1. VH-hIgGI CHI-CH3 LALAPG
(constant heavy chain)-GS linker- Rspo2RA
(SEQ ID NO: 14)
RAEVQLVESGGGLVQPGGSLRLSCAASGFTFTSYYISWVRQAPGKGLEWVAEISPYS
GSTYYADSVKGRFTISADTSKNTAYLQMNSLRAEDTAVYYCALRARPPIRLHPRGSV
MDYWGQGTLVTVSSGGGGGGGGSGGGGSEVQLVESGGGLVQPGGSLRLSCAASGFNI
SSSYIHWVRQAPGKGLEWVAYIYSSYGSTYYADSVKGRFTISADTSKNTAYLQMNSLR
AEDTAVYYCARASWYALDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSN
TKVDKKVEPKSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALGAPIEKTISKAKGQP
REPQVYTLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGKGGGGSGSGGSGGGGSNPICKGCLSCSKDNGC
SRCQQKLFFFLRREGMRQYGECLHSCPSGYYGHRAPDMNRCARCRIENCDSCRSKDACTKCKV
GFYLHRGRCFDECPDGFAPLEETMECVE
17SB9-03-HC1
Format: VHH03hIgG1 Fc LALAPG(Hole) -GS linker-17SB9-GS linker-6xHis
(SEQ ID NO: 15)
DVQLVESGGGLVQPGGSLRLSCTSSANINSIETLGWYRQAPGKQRELIANMRGG
GYMKYAGSLKGRFTMSTESAKNTMYLQMNSLKPEDTAVYYCYVKLRDDDYV
YRGQGTQVTVSSDKTHTCPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALGAPIEKTISKAKG
QPREPQVYTLPPSREEMTKNQVSLSCAVKGFYPSDIAVEWESNGQPENNYKTTPPVLDSDGSFFLVSKL
TVDKSRWQQGNVFSCSVMHEALHNHYTQKSLSLSPGKGGSGSAVQQVESGGGLVQPGGSLR
LSCAASGFTLDYYAISWFRQAPGKKRELVADITSGGSTNYADSVKGRFTISRDNA
KNTIYLQMNSLKPEDTAVYFCNAVTYNGYTIWGQGTQVTVSSGSGS [symbol]
17SB9-03-HC2
Format: 17SB9_hlgG1_Fc LALAPG(Knob) -GS linker-VHH03-GS linker-Flag
(SEQ ID NO: 16)
AVQQVESGGGLVQPGGSLRLSCAASGFTLDYYAISWFRQAPGKKRELVADITSG
GSTNYADSVKGRFTISRDNAKNTIYLQMNSLKPEDTAVYFCNAVTYNGYTIWG
QGTQVTVSSDKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALGAPIEKTISKAKGQPRE
PQVYTLPPSREEMTKNQVSLWCLVKGFYPSDIAVEWESNGQPENNYKTTPPVLDSDGSFFLYSKLTVD
KSRWQQGNVFSCSVMHEALHNHYTQKSLSLSPGKGGSGSDVQLVESGGGLVQPGGSLRLSC
TSSANINSIETLGWYRQAPGKQRELIANMRGGGYMKYAGSLKGRFTMSTESAK
NTMYLQMNSLKPEDTAVYYCYVKLRDDDYVYRGQGTQVTVSSGSGSG

[symbol]

[symbol]

In particular embodiments, the engineered WNT agonist comprises one or more, e.g., two, sequence having at least 90%, at least 95%, at least 98%, at least 99%, or 100% identity to a sequence set forth in SEQ ID NO:1, and one or more, e.g., two, sequence having at least 90%, at least 95%, at least 98%, at least 99%, or 100% identity to a sequence set forth in SEQ ID NO:2. In particular embodiments, the engineered WNT agonist comprises one or more, e.g., two, sequence having at least 90%, at least 95%, at least 98%, at least 99%, or 100% identity to a sequence set forth in SEQ ID NO:3, and one or more, e.g., two, sequence having at least 90%, at least 95%, at least 98%, at least 99%, or 100% identity to a sequence set forth in SEQ ID NO:4. In particular embodiments, the engineered WNT agonist comprises one or more, e.g., two, sequence having at least 90%, at least 95%, at least 98%, at least 99%, or 100% identity to a sequence set forth in SEQ ID NO:5, and one or more, e.g., two, sequence having at least 90%, at least 95%, at least 98%, at least 99%, or 100% identity to a sequence set forth in SEQ ID NO:6. In particular embodiments, the engineered WNT agonist comprises two sequences having at least 90%, at least 95%, at least 98%, at least 99%, or 100% identity to a sequence set forth in SEQ ID NO:1, and two sequences having at least 90%, at least 95%, at least 98%, at least 99%, or 100% identity to a sequence set forth in SEQ ID NO:2. In particular embodiments, the engineered WNT agonist comprises two sequences having at least 90%, at least 95%, at least 98%, at least 99%, or 100% identity to a sequence set forth in SEQ ID NO:3, and two sequences having at least 90%, at least 95%, at least 98%, at least 99%, or 100% identity to a sequence set forth in SEQ ID NO:4. In particular embodiments, the engineered WNT agonist comprises two sequences having at least 90%, at least 95%, at least 98%, at least 99%, or 100% identity to a sequence set forth in SEQ ID NO:5, and two sequences having at least 90%, at least 95%, at least 98%, at least 99%, or 100% identity to a sequence set forth in SEQ ID NO:6. In particular embodiments, each sequence comprises less than one (i.e., zero), less than two, less than three, less than four, less than five, less than six, less than seven, less than eight, less than nine, less than ten, less than eleven, or less than twelve modifications, e.g., substitutions, of amino acids present within CDRs of the WNT agonist. In particular embodiments, the engineered WNT agonist comprises less than one (i.e., zero), less than two, less than three, less than four, less than five, less than six, less than seven, less than eight, less than nine, less than ten, less than eleven, or less than twelve modifications of amino acids present within CDRs of the WNT agonist. In certain embodiments, the engineered Wnt agonist comprises an antibody or antigen-binding fragment thereof, wherein the antibody or antigen-binding fragment thereof comprises at least two, at least three, at least four, at least five, or at least six CDRs of any of the sequences of SEQ ID NOs: 1-16.

In some embodiments, the engineered WNT agonist comprises two sequences having at least 90%, at least 95%, at least 98%, at least 99%, or 100% identity to a sequence set forth in SEQ ID NO:7, and two sequences having at least 90%, at least 95%, at least 98%, at least 99%, or 100% identity to a sequence set forth in SEQ ID NO:8. In particular embodiments, each sequence comprises less than one (i.e., zero), less than two, less than three, less than four, less than five, less than six, less than seven, less than eight, less than nine, less than ten, less than eleven, or less than twelve modifications, e.g., substitutions, of amino acids present within CDRs of the WNT agonist. In particular embodiments, the engineered WNT agonist comprises less than one (i.e., zero), less than two, less than three, less than four, less than five, less than six, less than seven, less than eight, less than nine, less than ten, less than eleven, or less than twelve modifications of amino acids present within CDRs of the WNT agonist, e.g., less than two, less than three, less than four, less than five, less than six, less than seven, less than eight, less than nine, less than ten, less than eleven, or less than twelve amino acid substitutions within CDRs of the WNT agonist. In certain embodiments, the engineered Wnt agonist comprises an antibody or antigen-binding fragment thereof, wherein the antibody or antigen-binding fragment thereof comprises at least two, at least three, at least four, at least five, or at least six CDRs of any of the sequences of SEQ ID NOs: 1-16.

In particular embodiments, the engineered WNT agonist comprises one or more, e.g., two, sequence having at least 90%, at least 95%, at least 98%, at least 99%, or 100% identity to a sequence set forth in SEQ ID NO:9, and one or more, e.g., two, sequence having at least 90%, at least 95%, at least 98%, at least 99%, or 100% identity to a sequence set forth in SEQ ID NO:10. In particular embodiments, the engineered WNT agonist comprises one or more, e.g., two, sequence having at least 90%, at least 95%, at least 98%, at least 99%, or 100% identity to a sequence set forth in SEQ ID NO:11, and one or more, e.g., two, sequence having at least 90%, at least 95%, at least 98%, at least 99%, or 100% identity to a sequence set forth in SEQ ID NO:12. In particular embodiments, the engineered WNT agonist comprises one or more, e.g., two, sequence having at least 90%, at least 95%, at least 98%, at least 99%, or 100% identity to a sequence set forth in SEQ ID NO:13, and one or more, e.g., two, sequence having at least 90%, at least 95%, at least 98%, at least 99%, or 100% identity to a sequence set forth in SEQ ID NO:14. In particular embodiments, the engineered WNT agonist comprises one or more, e.g., two, sequence having at least 90%, at least 95%, at least 98%, at least 99%, or 100% identity to a sequence set forth in SEQ ID NO:15, and one or more, e.g., two, sequence having at least 90%, at least 95%, at least 98%, at least 99%, or 100% identity to a sequence set forth in SEQ ID NO:16. In particular embodiments, each sequence comprises less than one (i.e., zero), less than two, less than three, less than four, less than five, less than six, less than seven, less than eight, less than nine, less than ten, less than eleven, or less than twelve modifications of amino acids present within CDRs of the WNT agonist. In particular embodiments, the engineered WNT agonist comprises less than one (i.e., zero), less than two, less than three, less than four, less than five, less than six, less than seven, less than eight, less than nine, less than ten, less than eleven, or less than twelve modifications of amino acids present within CDRs of the WNT agonist, e.g., less than two, less than three, less than four, less than five, less than six, less than seven, less than eight, less than nine, less than ten, less than eleven, or less than twelve amino acid substitutions within CDRs of the WNT agonist. In certain embodiments, the engineered Wnt agonist comprises an antibody or antigen-binding fragment thereof, wherein the antibody or antigen-binding fragment thereof comprises at least two, at least three, at least four, at least five, or at least six CDRs of any of the sequences of SEQ ID NOs: 1-16.

### III. Pharmaceutical Compositions

Pharmaceutical compositions comprising a WNT antagonist molecule or WNT agonist molecule (a WNT antagonist/agonist molecule) described herein and one or more pharmaceutically acceptable diluent, carrier, or excipient are also disclosed. The term “WNT antagonist/agonist molecule” refers collectively to both WNT agonist molecules and WNT agonist molecules.

In further embodiments, pharmaceutical compositions comprising a polynucleotide comprising a nucleic acid sequence encoding a WNT antagonist/agonist molecule described herein and one or more pharmaceutically acceptable diluent, carrier, or excipient are also disclosed. In certain embodiments, the polynucleotides are DNA or mRNA, e.g., a modified mRNA. In particular embodiments, the polynucleotides are modified mRNAs further comprising a 5′ cap sequence and/or a 3′ tailing sequence, e.g., a polyA tail. In other embodiments, the polynucleotides are expression cassettes comprising a promoter operatively linked to the coding sequences.

In some embodiments the WNT antagonist/agonist is an engineered recombinant polypeptide incorporating various epitope binding fragments that bind to various molecules in the WNT signaling pathway. For example, a WNT antagonist can be an antibody or fragment thereof that binds to a FZD receptor and/or an LRP receptor and inhibits WNT signaling. The FZD and LRP antibody fragments (e.g., Fab, scFv, VHH/sdAbs, etc.) may be joined together directly or with various size linkers, on one molecule.

Conversely, engineered WNT agonists/antagonists can also be recombinant polypeptides incorporating epitope binding fragments that bind to various molecules in the WNT signaling pathway and enhance WNT signaling. For example, a WNT agonist can be an antibody or fragment thereof that binds to Fzd receptor and/or an LRP receptor and enhances WNT signaling. The Fzd and LRP antibody fragments (e.g., Fab, scFv, VHH/sdAbs, etc.) may be joined together directly or with various size linkers, on one molecule.

In further embodiments, pharmaceutical compositions comprising an expression vector, e.g., a viral vector, comprising a polynucleotide comprising a nucleic acid sequence encoding a WNT antagonist/agonist molecule described herein and one or more pharmaceutically acceptable diluent, carrier, or excipient are also disclosed. In certain embodiments, the nucleic acid sequence encoding the WNT antagonist molecule and the nucleic acid sequence encoding the WNT agonist are in the same polynucleotide, e.g., expression cassette.

The present disclosure further contemplates a pharmaceutical composition comprising a cell comprising an expression vector comprising a polynucleotide comprising a promoter operatively linked to a nucleic acid encoding a WNT antagonist/agonist molecule and one or more pharmaceutically acceptable diluent, carrier, or excipient. In particular embodiments, the pharmaceutical composition further comprises a cell comprising an expression vector comprising a polynucleotide comprising a promoter operatively linked to a nucleic acid sequence encoding a WNT antagonist and a WNT agonist. In certain embodiments, the nucleic acid sequence encoding the WNT antagonist molecule and the nucleic acid sequence encoding the WNT agonist molecule are present in the same polynucleotide, e.g., expression cassette and/or in the same cell. In particular embodiments, the cell is a heterologous cell, or an autologous cell obtained from the subject to be treated.

In particular embodiments, the cell is a stem cell, e.g., an adipose-derived stem cell or a hematopoietic stem cell. The present disclosure contemplates pharmaceutical compositions comprising a first molecule for delivery of a WNT antagonist molecule as a first active agent, and a WNT agonist as a second molecule. The first and second molecule may be the same type of molecule or different types of molecules. For example, in certain embodiments, the first and second molecule may each be independently selected from the following types of molecules: polypeptides, small organic molecules, nucleic acids encoding the first or second active agent (optionally DNA or mRNA, optionally modified RNA), vectors comprising a nucleic acid sequence encoding the first or second active agent (optionally expression vectors or viral vectors), and cells comprising a nucleic acid sequence encoding the first or second active agent (optionally an expression cassette).

The subject molecules, alone or in combination, can be combined with pharmaceutically acceptable carriers, diluents, excipients and reagents useful in preparing a formulation that is generally safe, non-toxic, and desirable, and includes excipients that are acceptable for mammalian, e.g., human or primate, use. Such excipients can be solid, liquid, semisolid, or, in the case of an aerosol composition, gaseous. Examples of such carriers, diluents and excipients include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; penetration-enhancing agents such as sodium caprate, viscosity-enhancing agents such as hydroxypropyl methylcellulose, in particular for eyedrop formulations, and compounds for the adjustment of tonicity such as sodium chloride or dextrose. The pH can be adjusted with acids or bases, such as hydrochloric acid or sodium hydroxide. In particular embodiments, the pharmaceutical compositions are sterile.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid such that it can be drawn into a syringe or delivered to a subject from a syringe. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the WNT antagonist/agonist antibody or antigen-binding fragment thereof (or encoding polynucleotide or cell comprising the same) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the antibody or antigen-binding fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art.

It may be advantageous to formulate the pharmaceutical compositions in dosage unit form for ease of administration and uniformity of dosage. Dosage unit form as used herein refers to physically discrete units suited as unitary dosages for the subject to be treated; each unit containing a predetermined quantity of active antibody or antigen-binding fragment thereof calculated to produce the desired therapeutic effect in association with the required pharmaceutical carrier. The specification for the dosage unit forms are dictated by and directly dependent on the unique characteristics of the antibody or antigen-binding fragment thereof and the particular therapeutic effect to be achieved, and the limitations inherent in the art of compounding such an active antibody or antigen-binding fragment thereof for the treatment of individuals.

The pharmaceutical compositions can be included in a container, pack, or dispenser, e.g., syringe or eye dropper, e.g., a prefilled syringe or eye dropper, together with instructions for administration.

The pharmaceutical compositions of the present disclosure may be delivered to a subject in the form of a pill, capsule, cream, salve, syrup, dermal patch, suppository, intravenous drip, aqueous solution, non-aqueous solution, eye wash solution, or any combination of thereof.

The pharmaceutical compositions of the present disclosure may be delivered to a subject by direct ophthalmic application, muscular injection, intravenous injection, peritoneal injection, nasally, orally, rectally, or any combination thereof.

The pharmaceutical compositions of the present disclosure encompass any pharmaceutically acceptable salts, esters, or salts of such esters, or any other compound which, upon administration to an animal comprising a human, is capable of providing (directly or indirectly) the biologically active antibody or antigen-binding fragment thereof.

The present disclosure includes pharmaceutically acceptable salts of a WNT antagonist/agonist molecule described herein. The term “pharmaceutically acceptable salt” refers to physiologically and pharmaceutically acceptable salts of the compounds of the present disclosure: i.e., salts that retain the desired biological activity of the parent compound and do not impart undesired toxicological effects thereto. A variety of pharmaceutically acceptable salts are known in the art and described, e.g., in “Remington's Pharmaceutical Sciences”, 17th edition, Alfonso R. Gennaro (Ed.), Mark Publishing Company, Easton, PA, USA, 1985 (and more recent editions thereof), in the “Encyclopedia of Pharmaceutical Technology”, 3rd edition, James Swarbrick (Ed.), Informa Healthcare USA (Inc.), NY, USA, 2007, and in J. Pharm. Sci. 66:2 (1977). Also, for a review on suitable salts, see “Handbook of Pharmaceutical Salts: Properties, Selection, and Use” by Stahl and Wermuth (Wiley-VCH, 2002). Pharmaceutically acceptable base addition salts are formed with metals or amines, such as alkali and alkaline earth metals or organic amines.

Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure.

In some embodiments, the pharmaceutical composition provided herein comprise a therapeutically effective amount of a WNT antagonist/agonist molecule or pharmaceutically acceptable salt thereof in admixture with a pharmaceutically acceptable carrier, diluent and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer may be in the range of 6.5 to 7.75, preferably 7 to 7.5, and most preferably 7.2 to 7.4.

### IV. Methods of Use

The present disclosure also provides methods for using the WNT antagonist/agonist molecules, e.g., to modulate a WNT signaling pathway, e.g., to increase or decrease WNT signaling, and the administration of a WNT antagonist/agonist molecule in a variety of therapeutic settings. Provided herein are methods of treatment using a WNT antagonist/agonist molecule. In one embodiment, a WNT antagonist/agonist molecule is provided to a subject having a disease involving inappropriate or deregulated WNT signaling.

In certain embodiments, a WNT antagonist/agonist molecule may be used to block or enhance a WNT signaling pathway in a tissue or a cell. Antagonizing the WNT signaling pathway may include decreasing or inhibiting WNT signaling in a cell or tissue. Agonizing the WNT signaling pathway may include, for example, increasing WNT signaling or enhancing WNT signaling in a tissue or cell. Thus, in some aspects, the present disclosure provides a method for antagonizing/agonizing a WNT signaling pathway in a cell, comprising contacting the tissue or cell with an effective amount of a WNT antagonist/agonist molecule or pharmaceutically acceptable salt thereof disclosed herein, wherein the WNT antagonist/agonist molecule is a WNT signaling pathway antagonist/agonist. In some embodiments, contacting occurs in vitro, ex vivo, or in vivo. In particular embodiments, the cell is a cultured cell, and the contacting occurs in vitro.

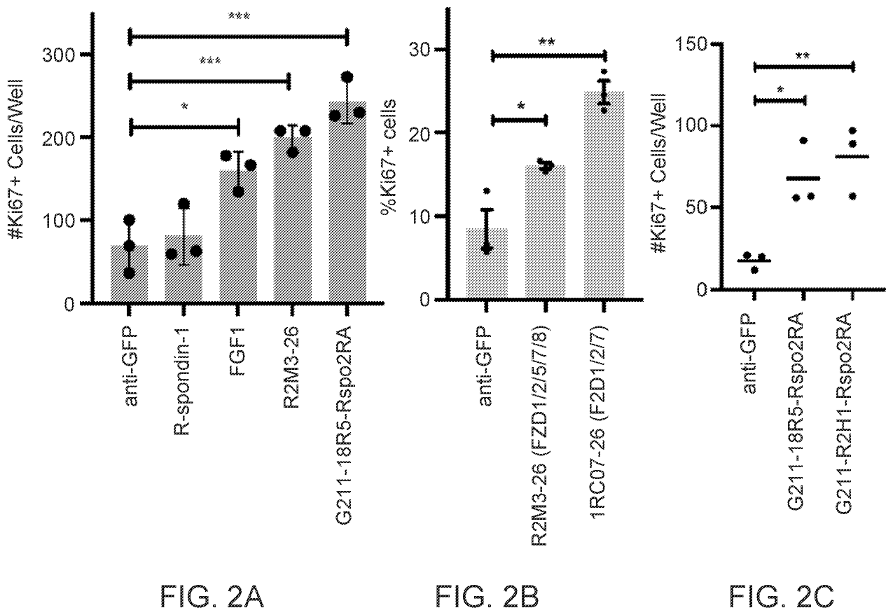

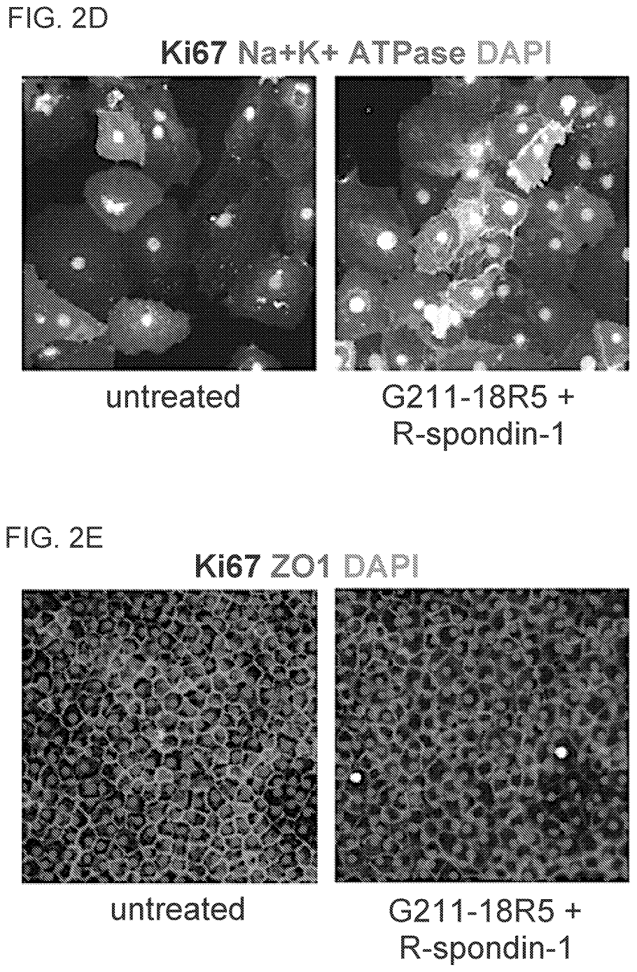

The WNT antagonist/agonist molecules may be used for the treatment of keratopathy. In particular, experiments have indicated that activation of WNT signaling regulates cornea cell growth and layer patterning. (Zhang et al., Development, 2015 142:3383-93.) Conditional knockout of either β-catenin or Lrp5/6 results in mis-stratification, particularly of epithelium. Recent in vitro experimentation indicates that WNT agonists promote cell growth in homogenized cornea cell culture. See, e.g., FIGS. 2A-E and 7A-B. Therefore, controlled administrations of WNT agonist or/and antagonist are proposed to promote epithelial and endothelial cell growth, and reverse pathology of various keratopathies, including dystrophies and scarring. In the particular embodiments, WNT agonist/antagonist will be administered in either earlier or later phase of keratopathy disease progression in the subjects.

Both WNT agonists and antagonists may be administered alone as a monotherapy or sequentially. In certain embodiments, the method comprises administration of a WNT agonist in earlier phase of disease development, followed by administration of a WNT antagonist at late stages of the disease where there is significant scarring or fibrosis.

The present disclosure also provides for combination treatment with known treatments for keratopathies. Examples of therapies that could be administered with a WNT agonist or antagonist include, but are not limited to, Rho kinase inhibitors (e.g., Ripasudil or Fasudil), anti-inflammatories (e.g., steroidal and non-steroidal), and immunosuppressants.

Keratopathies that may be treated include, but are not necessarily limited to, bacterial infections, viral infections (e.g., herpes simplex keratitis and herpes zoster ophthalmicus (i.e., shingles)), fungal infections, protozoan infections, archaeal infections, genetic disorders, brittle cornea syndrome (BCS), corneal endothelial-mesenchymal transition (EnMT), corneal epithelial-mesenchymal transition (EMT), corneal fibrosis, Cogan syndrome, corneal ulcer, epithelial basement membrane dystrophy (EBMD, ABMD, Map Dot), Fleck corneal dystrophy, Fuchs' dystrophy (also called Fuchs' corneal endothelial dystrophy (FCED) or Fuchs' endothelial dystrophy (FED) or Fuchs' endothelial cell dystrophy (FECD)), gelatinous droplike corneal dystrophy, granular corneal dystrophy type I, granular corneal dystrophy type II, interstitial keratitis, iridocorneal endothelial syndrome (ICE), keratoconjunctivitis sicca, keratoconus, keratomalacia, lattice dystrophy type I, lattice dystrophy type II, Lisch corneal dystrophy, macular corneal dystrophy, Meesmann corneal dystrophy, peripheral ulcerative keratitis, phlyctenular keratoconjunctivitis, posterior polymorphous corneal dystrophy, pterygium, Reis-Bückler corneal dystrophy, Schnyder crystalline corneal dystrophy, Stevens-Johnson syndrome, superficial punctate keratitis, Thiel-Behnke corneal dystrophy, neurotrophic keratitis, herpetic keratitis and scarring.

In a further embodiment, the WNT antagonist and/or WNT agonist molecule may also incorporate a tissue targeting moiety, e.g., an antibody or fragment thereof that recognizes a cornea tissue or cell type specific receptor or cell surface molecule.

The dose and dosage regimen may depend upon a variety of factors readily determined by a physician, such as the nature of the disease or disorder, the characteristics of the subject, and the subject's history. In particular embodiments, the amount of a Wnt signaling molecule administered or provided to the subject is in the range of about 0.01 mg/kg to about 50 mg/kg, 0.1 mg/kg to about 500 mg/kg, or about 0.1 mg/kg to about 50 mg/kg of the subject's body weight. In particular embodiments, the amount of an engineered WNT signaling modulator administered or provided to the subject is in the range of about 0.01 mg/kg to about 50 mg/kg, about 0.1 mg/kg to about 500 mg/kg, or about 0.1 mg/kg to about 50 mg/kg of the subject's body weight. In certain embodiments of any of the methods disclosed herein, the WNT signaling modulator is administered to a subject, e.g., a mammal, intravenously, e.g., as a bolus injection, or subcutaneously. In particular embodiments, the WNT signaling modulator is administered at least once per week. In particular embodiments, the subject is administered about 0.5 to about 100 mg/kg body weight of the WNT signaling modulator, or about 2 to about 50 mg/kg body weight of the WNT agonist, e.g., about 2 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In particular embodiments, the subject is administered about 25 mg, about 75 mg, about 250 mg, about 750 mg, about 1500 mg or about 2250 mg of the WNT signaling modulator. In particular embodiments, the subject is administered about 3 to about 30 mg/kg body weight intravenously or subcutaneously at least once per week.

The therapeutic agent (e.g., a WNT antagonist/agonist) may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease. In some embodiments, the subject method results in a therapeutic benefit, e.g., preventing the development of a disorder, halting the progression of a disorder, reversing the progression of a disorder, etc. In some embodiments, the subject method comprises the step of detecting that a therapeutic benefit has been achieved. The ordinarily skilled artisan will appreciate that such measures of therapeutic efficacy will be applicable to the particular disease being modified and will recognize the appropriate detection methods to use to measure therapeutic efficacy.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the present disclosure have been described herein for purposes of illustration, various modifications may be made without deviating from the spirit and scope of the present disclosure. Accordingly, the present disclosure is not limited except as by the appended claims.

The broad scope of this disclosure is best understood with reference to the following example, which is not intended to limit the disclosure to a specific embodiment.

### EXAMPLES

### Example I. General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, glycosylation sites, and sequence alignments, are available. See, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

### Example II. Construction of WNT Mimetics

WNT mimetics R2M3-26, G211-18R5, 1RC07-26, 1SH1-03, and hp1SH1-03 were constructed as described in WO 2019/126398 A1, WO 2020/010308 A1, and WO 2020132365 A1, WO 2021173726, which are incorporated by reference in their entirety herein. All molecules are presented in Table 1. All recombinant proteins were produced in Expi293F™ cells (Thermo Fisher Scientific) by transient transfection unless otherwise specified. All IgG-based and Fc-containing constructs were first purified with Protein-A resin and eluted with 0.1 M glycine pH 3.5. All proteins were then polished by a size exclusion column in 2×HBS buffer (40 mM HEPES pH 7.4, 300 mM NaCl).

### Example III. WNT Agonist Treatment in Primary Human Corneal Endothelium Cell Culture

Human corneal endothelial cells with attached Descemet membrane were received from Eversight (Ann Arbor, MI). Multiple donor samples were pooled and digested in 2 mg/mL collagenase A in MEM-alpha+15% FBS+20 μg/mL gentamicin sulfate for 3 hours at 37° C. with occasional trituration. Samples were then washed with MEM-alpha+15% FBS+20 μg/mL gentamicin before resuspension and plating in Human Endothelial SFM+5% FBS+2 ng/mL EGF+20 μg/mL gentamicin sulfate.

Endothelial cells were treated with test proteins (5 nM R2M3-26, 1 μg/mL hR-spondin-1, 5 ng/mL FGF1, 5 nM G211-18R5-Rspo2RA, 5 nM G211-R2H1-Rspo2RA, or 5 nM 1RC07-26) or left untreated in Human Endothelial SFM+5% FBS+2 ng/mL EGF+20 μg/mL gentamicin sulfate. After 2 days, cells were fixed and immunofluorescence performed for Ki67 (Abcam #ab16667, 1:100) and Na+K+ ATPase (Sigma #05-369, 1:100) prior to nuclear staining with DAPI. Ki67 is a well-known marker for cell proliferation. See discussion in, e.g., Aung T N et al., Mod Pathol (2021), https://doi.org/10.1038/s41379-021-00745-6. Samples were imaged on a Leica Dmi8 microscope. The percentage of Ki67+ cells (# of Ki67+ cells/total # of cells) in the resulting images were quantified using a custom Python script. The results are depicted in FIGS. 2A, 2B, 2C, 2D, 2E.

### Example IV. WNT Agonist Treatments in Human Cornea Organ Culture

Human donor cornea with Fuchs' dystrophy was obtained from Eversight Eye Bank. Tissue was cut into pieces and cultured in Human Endothelial SFM+5% FBS+anti-mycotic/anti-biotic for 3 days, with no treatment or Wnt agonist (5 nM G211-18R5+1 μg/mL R-spondin-1) treatment. Corneas were then fixed and immunofluorescence performed for proliferation marker Ki67 (Cell Signaling Technology #9449S, 1:400) and corneal endothelium marker ZO1 (Abcam #ab216880, 1:100). The results are depicted in FIG. 2D.

### Example V. WNT Agonist Treatments in Primary Human Corneal Epithelium Cell Culture

Primary human corneal epithelial cells (PCS-700-010™, lot #80915170) were received from American Type Culture Collection (ATCC®). (Cell product information sheet available at the time of filing at https://www.atcc.org/products/all/PCS-700-010.aspx, incorporated by reference in its entirety herein.) Cells were cultured according to ATCC® recommendations. (Available as of the time of filing at https://www.atcc.org/products/all/PCS-700-010.aspx#cultureconditions, incorporated by reference in its entirety herein.)

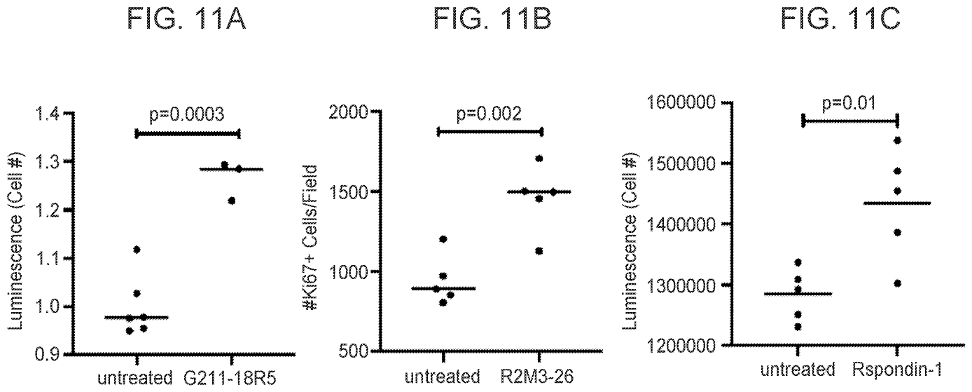

Cells were treated with WNT agonist (1 nM of R2M3-26) or left as untreated negative control for 2 days. Next, the cells were fixed, and immunofluorescence performed for Ki67 cell proliferation marker (Abcam #ab16667, 1:100) prior to nuclear staining with DAPI. Samples were imaged on a Leica Dmi8 microscope. #Ki67⁺ cells per field of view in the resulting images were quantified using a custom Python script. FIG. 11B.

Cells were treated with Wnt agonist (5 nM G211-18R5 or 1 μg/mL R-spondin-1) or left untreated for 2 days. After this, cell numbers were measured using a CellTiter-Glo® Viability Assay (Promega) according to the manufacturer's instructions. (Available as of the time of filing at https://www.promega.com/products/cell-health-assays/cell-viability-and-cytotoxicity-assays/celltiter glo-luminescent-cell-viability-assay/?catNum=G7570, incorporated by reference in its entirety herein.). Results are depicted in FIG. 11A and FIG. 11C.

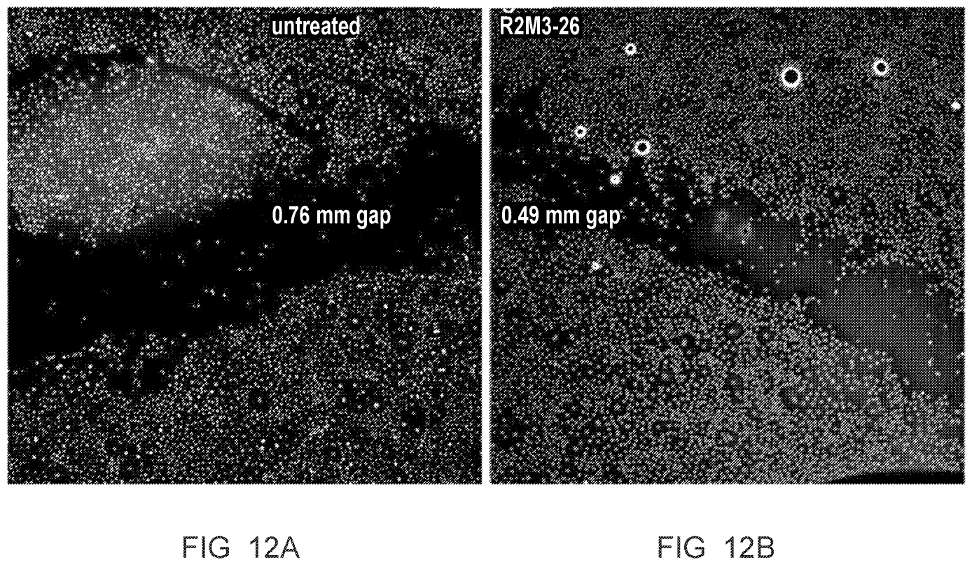

A wound healing assay kit was used (Abcam, ab242285) according to the manufacturer's instructions. Cells were left untreated or treated with Wnt agonist (1 nM R2M3-26). Results are depicted in FIG. 12A and FIG. 12B.

### Example VI. WNT Agonist Treatments in Rabbit Limbal Epithelial Organoid Culture

Rabbit eyes were received from Innovative Research. The limbal region was dissected and epithelial cells dissociated by incubation in 2.5 U/mL Dispase II in Organoid Basal Medium (Advanced DMEM/F12+× N-2+1× B-27+2 mM GlutaMAX+10 mM HEPES+1 mM N-acetylcysteine+1× penicillin-streptomycin+1× normocin). Epithelial cells were embedded in Matrigel and incubated with Organoid Basal Medium containing WNT agonist (5 nM R2M3-26) or 1 μg/mL R-spondin-1.

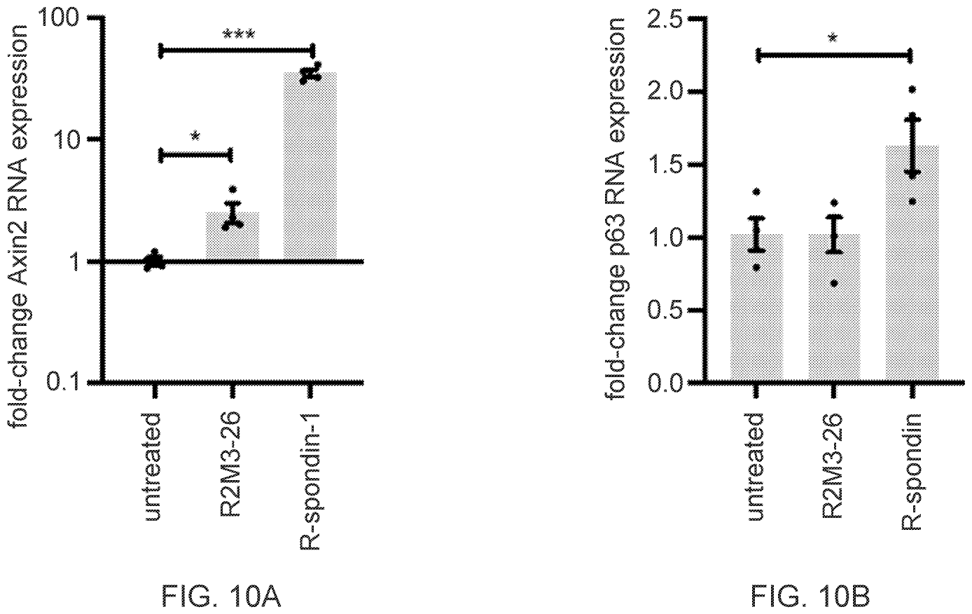

RNA was harvested from individual wells using an RNEasy Kit including RNeasy Plus Mini Kit (Qiagen) and cDNA synthesized using a SuperScript™ IV VILO™ cDNA Synthesis Kit (ThermoFisher). (Product information sheet available at the time of filing at https://www.thermofisher.com/document-connect/document-connect.html?url=https%3A%2F%2Fassets.thermofisher.com%2FTFS-Assets%2FLSG%2Fmanuals%2FsuperscriptIV_VILO_master_mix_UG.pdf&title=VXNlciB HdWlkZTogU3VwZXJTY3JpcHQgSVYgVklMTyBNYXN0ZXIgTW14, incorporated by reference herein in its entirety.) qPCR for Wnt target gene Axin2 and progenitor marker p63 was carried out using TaqMan™ Gene Expression Assay reagents (ThermoFisher) on a CFX96™ thermocycler (BioRad). β-actin was used for normalization. Results are depicted in FIG. 10A and FIG. 10B.

### Example VII. Lrp5/6, Fzd, and Znrf3/Rnf43 RNA Expression in Human Corneal Epithelium and Endothelium Tissue

A whole eye globe from a 47-year-old Caucasian female donor with no history of ocular disorders was received from Eversight (Ann Arbor, MI). The cornea was dissected, fixed in 10% formalin, and embedded in paraffin.

Fuchs' dystrophy tissue sample corneas were obtained from 73-year-old (Fuchs #1) and a 96-year-old (Fuchs #2), delivered from Eversight (Ann Arbor, MI). Fuchs #1 received a post-mortem diagnosis of Fuchs' dystrophy based on low endothelial cell counts and presence of severe guttate. Fuchs #2 received an in-life diagnosis of Fuchs' dystrophy. The corneas were dissected, fixed in 10% formalin, and embedded in paraffin.

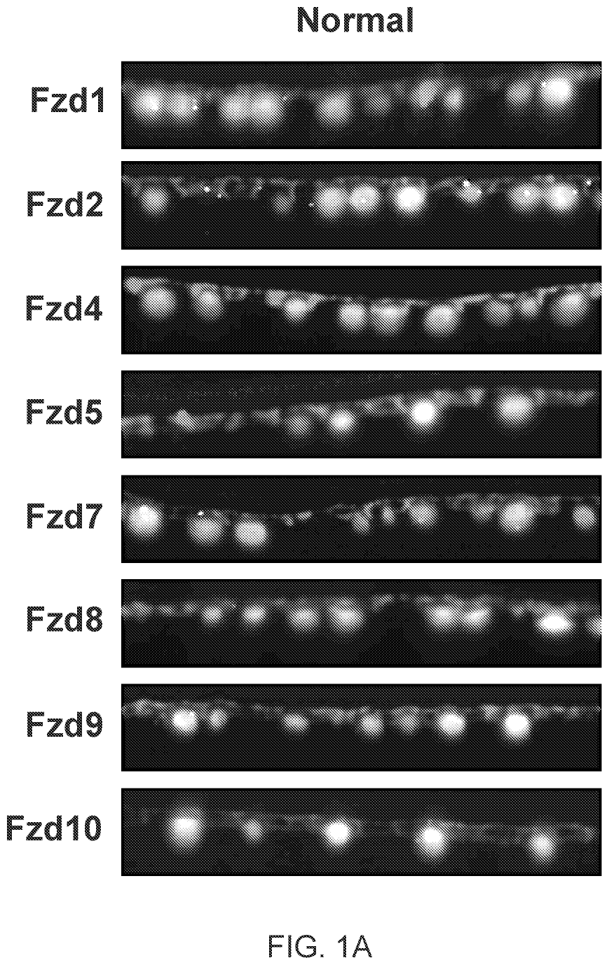

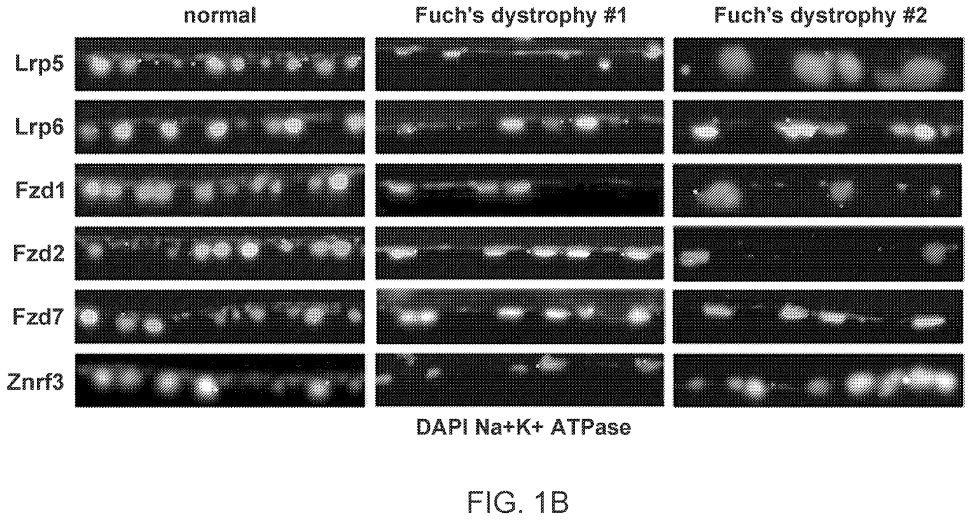

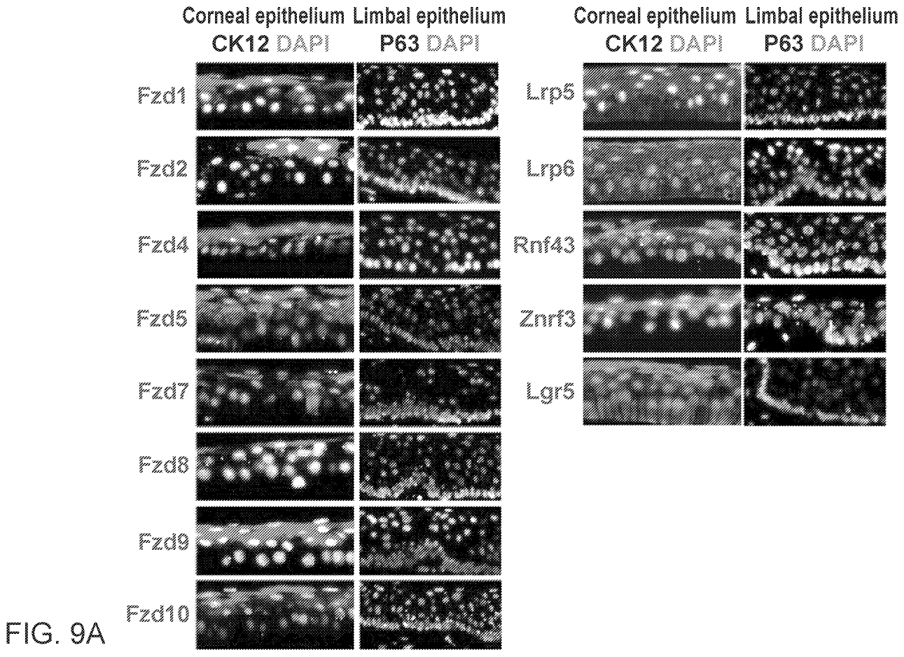

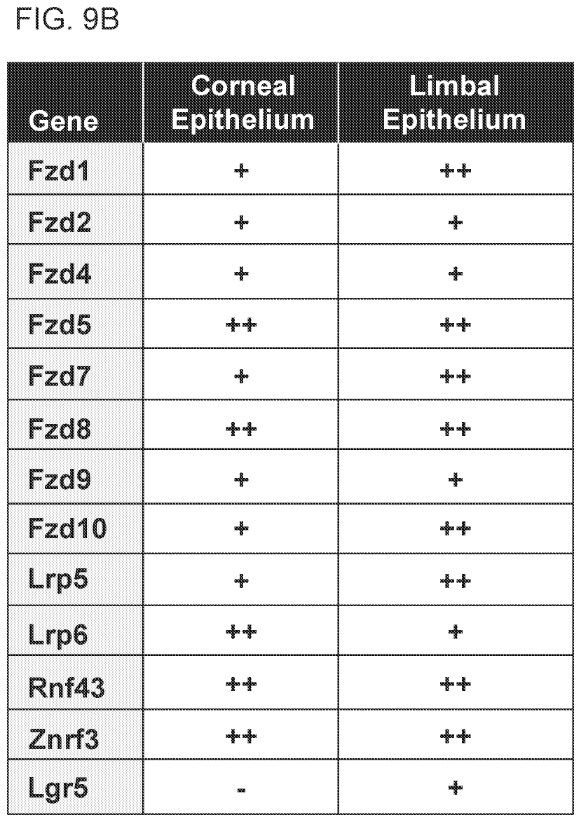

Sections of paraffin-embedded tissue were made and Lrp5/6, Fzd, and Znrf3/Rnf43 RNA levels measured using an RNAscope® kit including RNAscope Multiplex Fluorescent Reagent Kit v2 (ACDBio) according to the manufacturer's instructions. Immunofluorescence for the corneal epithelium marker CK12 (Abcam #ab185627, 1:100), limbal progenitor marker p63 (Biocare #4A4, 1:100) and corneal endothelium marker Na⁺K⁺ ATPase (ThermoFisher #05-369-MI, 1:100) was subsequently performed to confirm tissue identity. Stained sections were mounted in VECTASHIELD® mounting media with DAPI and imaged on a Leica DMi8A epifluorescence microscope. Results are depicted in FIG. 1 and FIG. 9.

### Example VIII. WNT Agonist Treatment in a Murine Limbus-to-Limbus Debridement Model

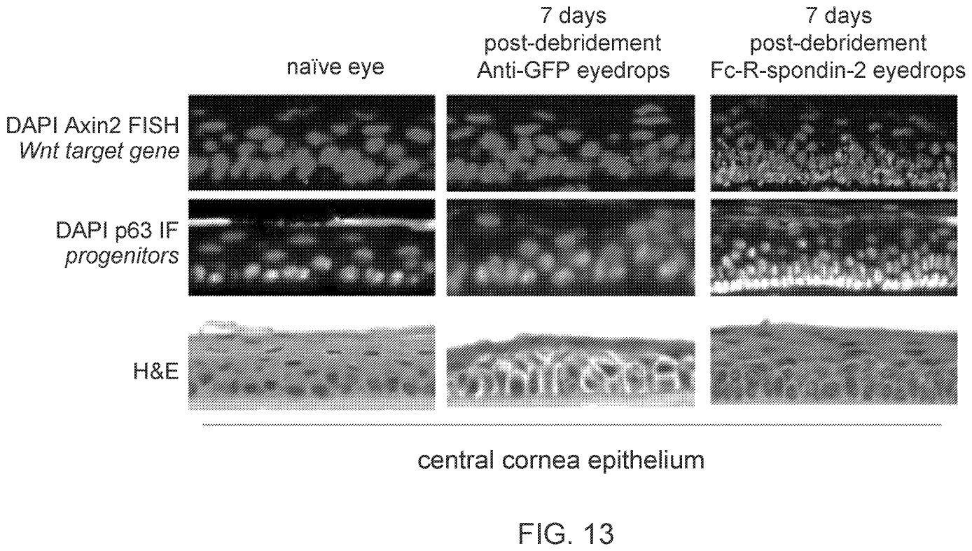

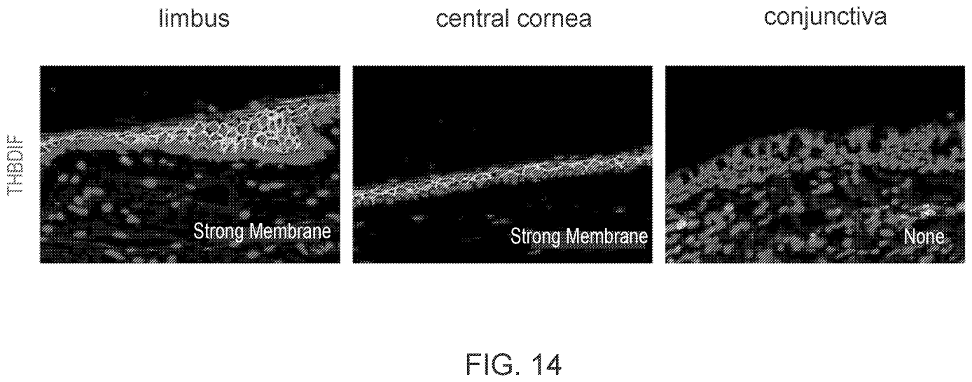

Mechanical debridement model: 6 to 12-week-old female C57Bl/6 mice were anesthetized by placing the animal in an induction chamber connected to an oxygen source and isoflurane vaporizer, and adjusting oxygen flow to 0.9 liters/min and the isoflurane vaporizer to 3-4%. As soon as the mouse became unresponsive and had shallow breathing, it was transferred to the anesthesia platform with the nose cone and placed on a heating pad. The head was positioned so that the eye to be injured is facing up towards the microscope objective. Only one eye per mouse was injured, the contralateral used as control. 1 drop of Proparacaine was placed on the cornea for 30 seconds. A Weck-cell™ cellulose eye spear was used to dry off the cornea by gently sweeping across cornea once and dabbing both corners of the eye. Periocular pressure was applied with one hand to proptose the mouse eye. The right cornea was marked with a 3 mm Trephine and scraped with a Algerbrush (Algerbrush II with 0.5 mm Burr (Katena, catalog number: K 2-4900) to remove the corneal epithelium, with care taken to avoid limbal blood vessels. After scraping, eyes were treated with erythromycin ophthalmic ointment to minimize inflammation and to keep the ocular surface moist while the mice were under anesthesia. Post surgery, the affected eye of the mice was treated with topical Fc-R-spondin-2 or Anti GFP antibody (5 mg/ml, 3 ul, 4×/day/7 days). At day 7, the mice were humanely euthanized and the eyes were entirely removed from each animal, and collected in Davidson's fixative, followed by 10% phosphate-buffered formalin and processed for histological analysis of Axin2, p63 and H&E as described in FIG. 13. The data indicate that R-spondin-2 eyedrops activate Wnt signaling, expand progenitor cells, thicken cornea epithelium and reduce conjunctivalization.

### Example IX. WNT Agonist Treatments in Naïve Mice by Intracameral and Intravitreal Injection

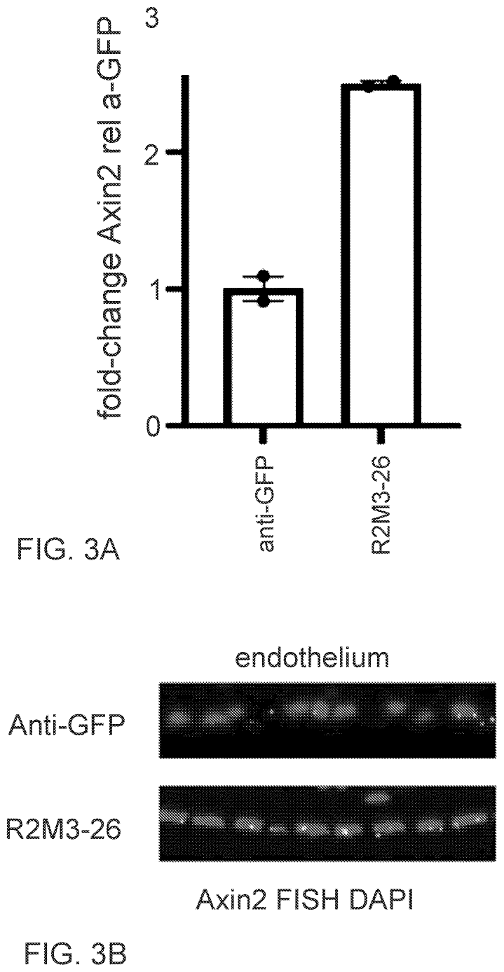

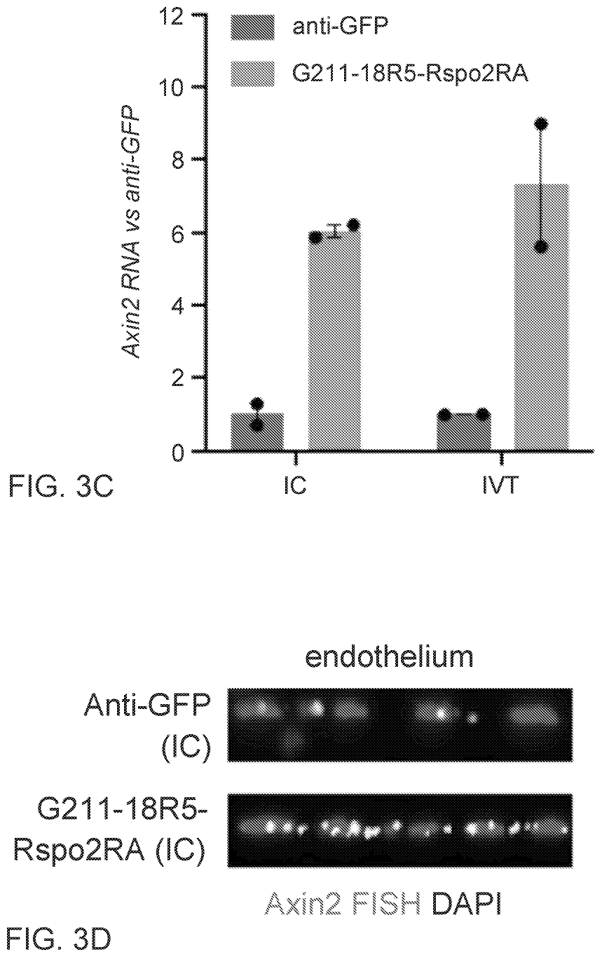

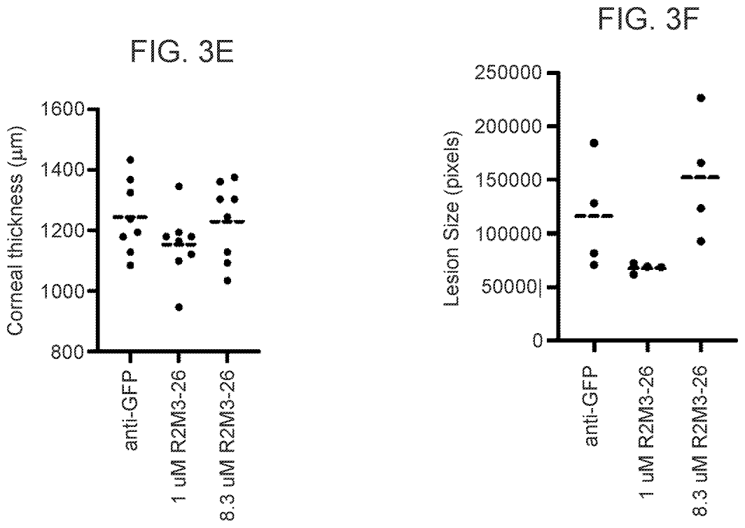

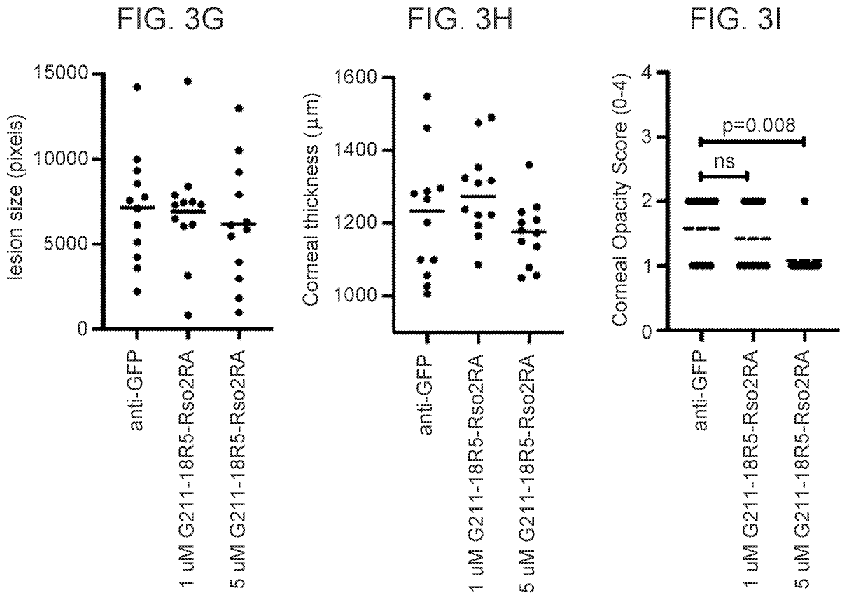

6 to 12-week-old female Balb/C mice were anesthetized by placing the animal in an induction chamber connected to an oxygen source and isoflurane vaporizer and adjusting oxygen flow to 0.9 liters/min and the isoflurane vaporizer to 3-4%. As soon as the mouse became unresponsive and had shallow breathing, it was transferred to the anesthesia platform with the nose cone. The head was positioned so that the eye to be injected is facing up towards the microscope objective. Only one eye per mouse was injected, the contralateral used as control. 1 drop of Proparacaine was placed on the cornea for 30 seconds. The cornea was punctured closely anterior to the iridocorneal angle. The fluid that emanates from the anterior chamber was wiped off with a weck-cell before 1 microliter of wnt agonist R2M3-26 or superagonist G211-18R5-Rspo2RA or anti-GFP antibody (2.6 ug) was injected into the anterior chamber (intracamerally) or posterior chamber (intravitreally) using a 33 gauge needle attached to Hamilton syringe. The mice were allowed to recover from the anesthesia. The mice were taken down the next day, the eyes harvested, and the corneas were dissected, fixed in 10% formalin, and embedded in paraffin. For half of the eye samples, sections of paraffin-embedded tissue were made and Axin2 levels measured using an RNAscope® kit including RNAscope Multiplex Fluorescent Reagent Kit v2 (ACDBio) according to the manufacturer's instructions. For the remaining eye samples, RT-qPCR for Wnt target gene Axin2 was carried out using TaqMan™ Gene Expression Assay reagents (ThermoFisher) on a CFX96™ thermocycler (BioRad). β-actin was used for normalization. Results from such studies are described in FIGS. 3A-3D.

### Example X. WNT Agonist Treatment in a Rabbit Endothelial Resection Model

The objective of these studies was to evaluate recovery of corneal endothelial cells (CECs) in the central cornea following removal by surgical scraping of endogenous CECs. The wnt agonists or control were injected intracamerally after resection surgery. Two separate studies were conducted to evaluate the ability of the WNT-mimetic R2M3-26, and the WNT superagonist G211-18R5-Rspo2RA in the rabbit endothelial resection model. In the first study, R2M3-26 at two different doses (1 and 8.3 uM) or anti-GFP antibody (8.3 uM) was administered on Day 0 after endothelial cell scraping to create a 10 mm lesion. The rabbits' eyes were examined at various timepoints including baseline and Day 3 post surgery for corneal thickness by optical coherence tomography, ocular exam to assess corneal opacity, and lesion size by performing corneal flat mounts and histologic staining for corneal endothelial markers. At day 3, lower dose R2M3-26 treatment trended towards decreased corneal thickness, lesion size and increased cell density (FIG. 3E and FIG. 3F). A second study was performed with the WNT superagonist G211-18R5-Rspo2RA at two concentrations (1 and 5 uM) and anti GFP (5 uM). A significant decrease in corneal opacity was observed in response to 5 uM wnt superagonist, whereas there was a trend of decreased corneal swelling/thickness and a reduction in lesion size in response to wnt superagonist at Day 3 (FIGS. 3G-3I).

### Example XI. UV-A Burn Model for Corneal Endothelial Damage (Liu et al., 2019, PNAS)

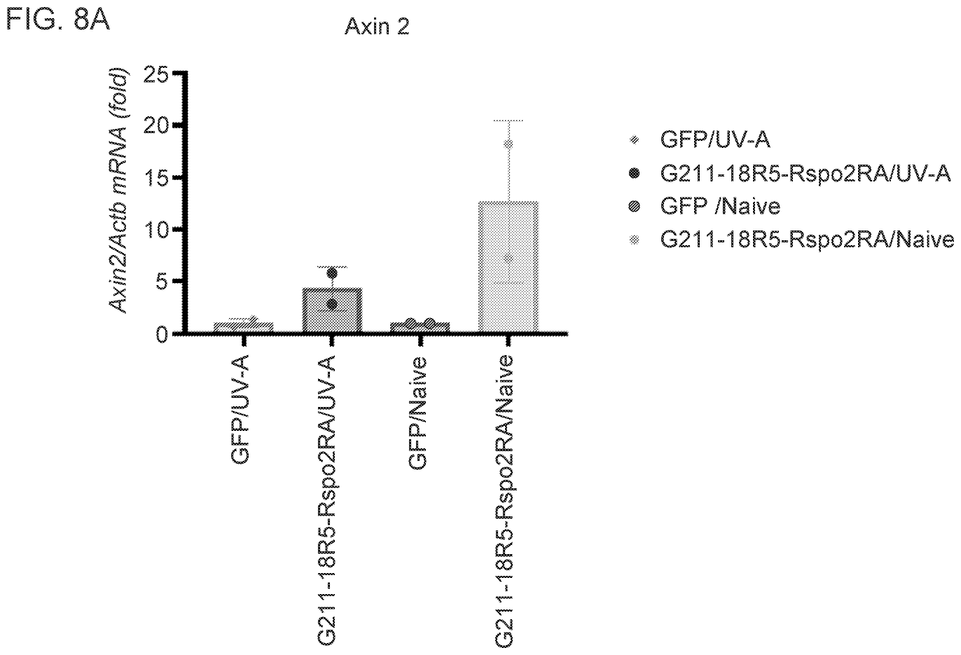

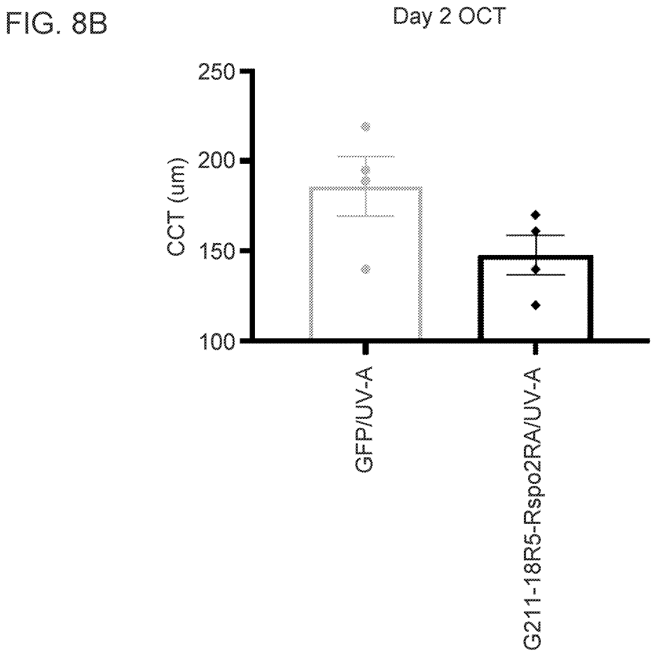

C57BL/J6 wild-type mice (7 to 15 wk old; The Jackson Laboratory) were used for this study. Mice were anesthetized with isoflurane. A UVA LED light source (M365LP1; Thorlabs) with an emission peak of 365 nm light, 9 nm bandwidth (FWHM) and irradiance of 398 mW/cm² was focused to illuminate a 4-mm-diameter spot onto the mouse cornea. The energy was measured using a laser sensor (model L49 [150A]; Ophir), and the time of UVA exposure was adjusted to deliver the appropriate fluence (20 min 57 s for 500 J/cm²). The right eye (OD) was irradiated while the contralateral eye (OS) was covered with heat retention drapes (SpaceDrapes, Inc.) to serve as untreated control. The drug was injected IVT a couple of hours before UV A burn. The cornea were imaged daily and photographed daily, and by OCT and at day 2, CCT was measured (FIG. 8A and FIG. 8B).

### Example XII. Cryoinjury Model

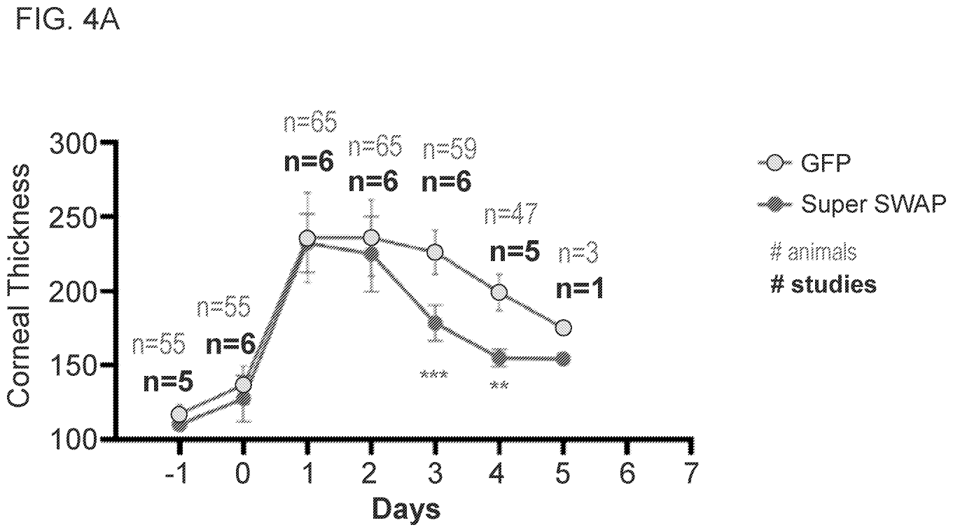

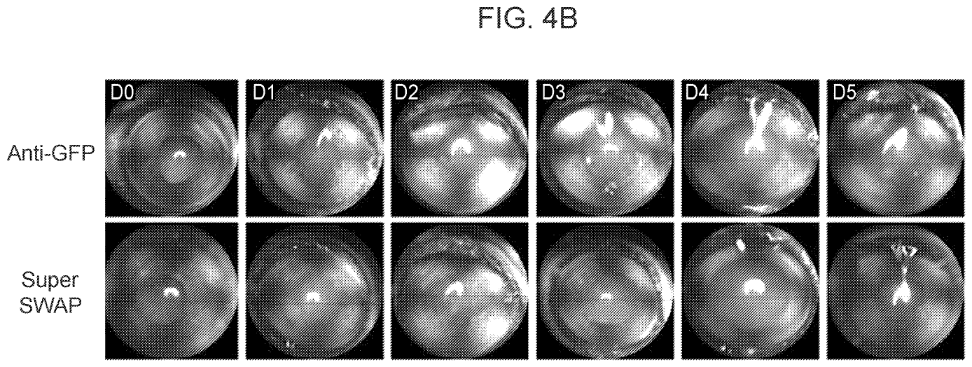

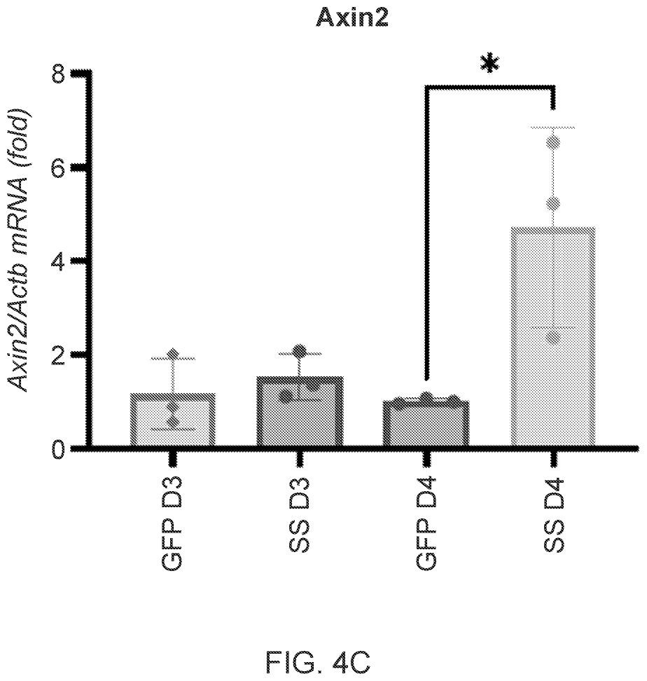

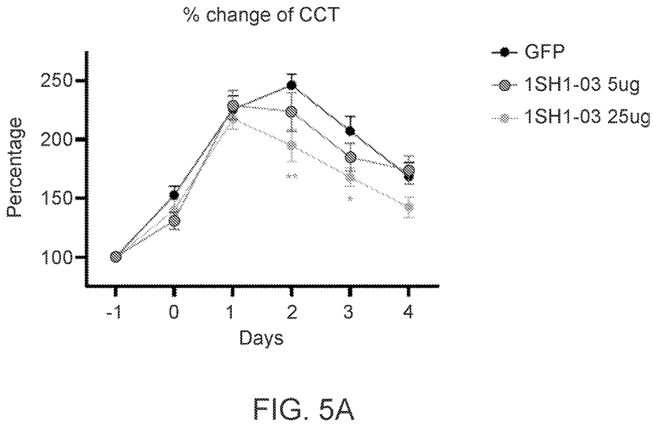

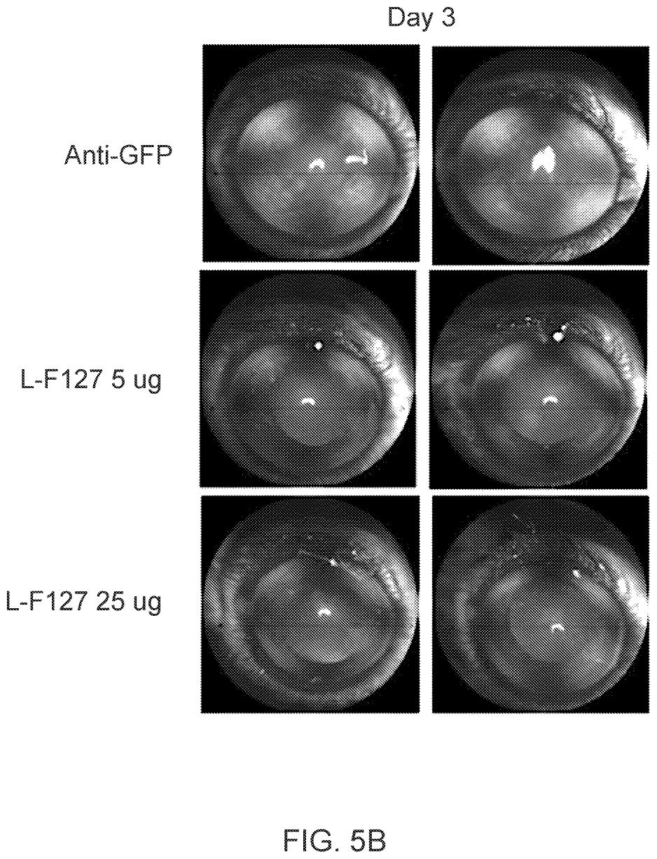

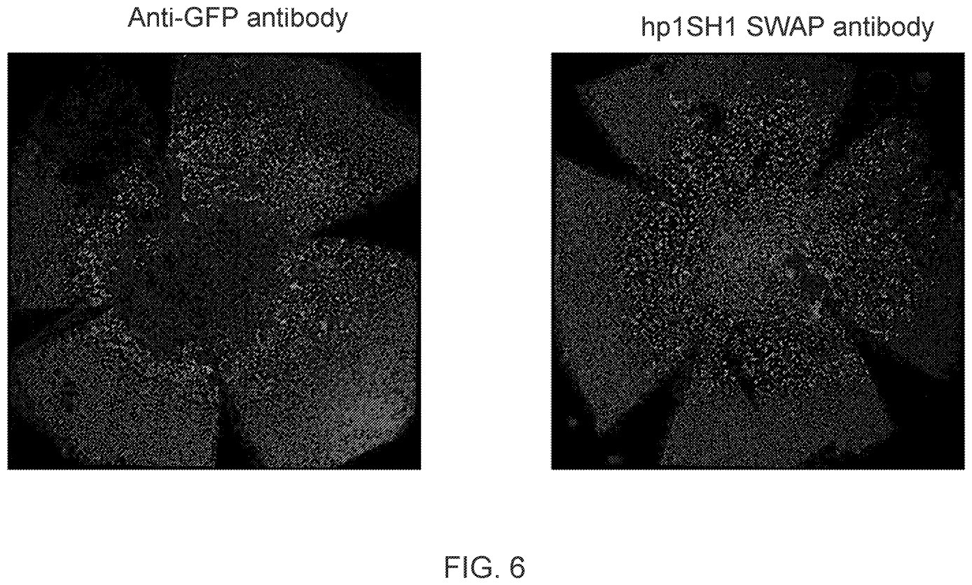

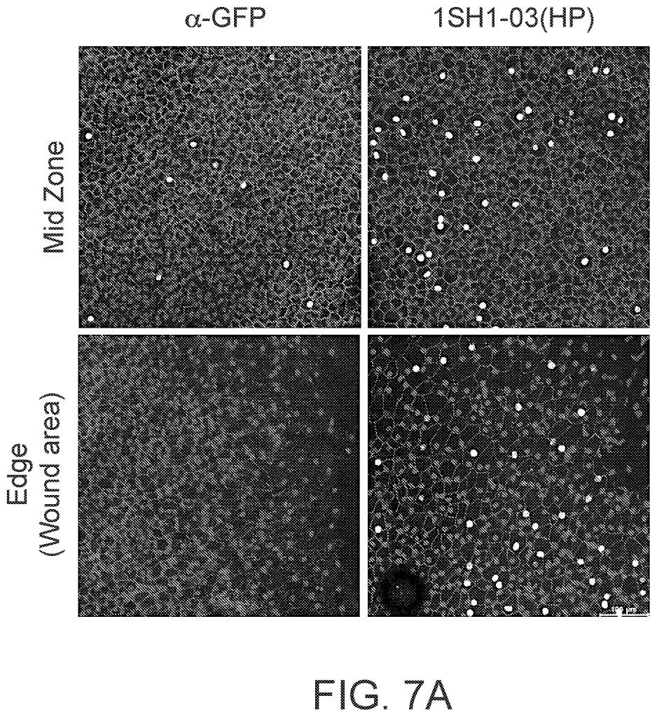

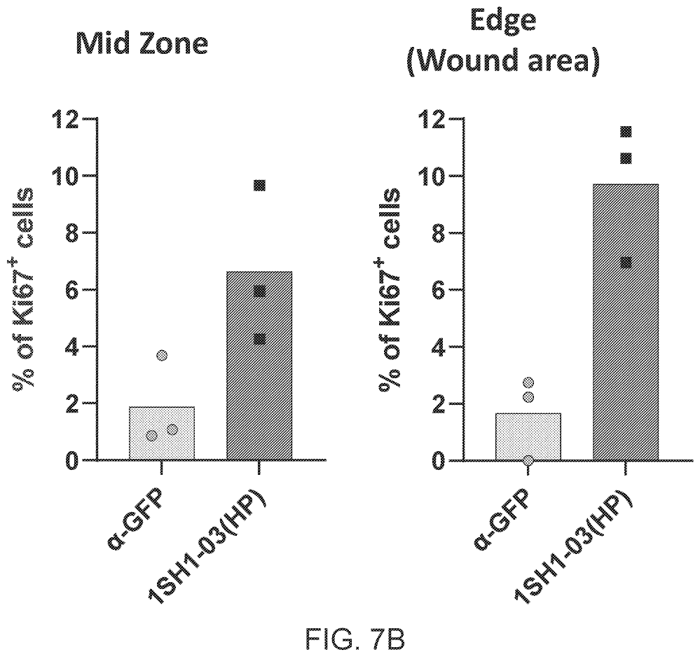

Mice were randomly divided into two groups, and 2 cycles of cryoinjury were applied to the right eye of each mouse (Han et al., 2013). Under Isoflurane anesthesia, transcorneal freezing was initiated by gently placing a cryoprobe made of stainless steel (2.5 mm in diameter; flat tip; ERBE Elektromedizin GmbH, Tilbingen, Germany), precooled in liquid nitrogen for a minute on the central cornea. A cryoprobe with a diameter of 2.5 mm was be chosen, as it is similar to the corneal diameter (2.6 mm) of C57BL/6 mice. No pressure was applied to avoid damage to adjacent tissue including the lens and the trabecular meshwork. The cryoprobe was kept on the corneal surface until an ice ball formed on the cornea and covered the entire corneal surface, which corresponded to the duration of 3 s, as the defect on the endothelium was shown to be the same size as the ice ball. This injury was repeated after a 1 min interval. Immediately after freezing, the cryoprobe was freed from the corneal surface with irrigation with a balanced salt solution, and the cornea was allowed to thaw spontaneously. Only the right eye was treated with the cryoprobe, and the left eye served as an untreated control. Recovery of the cornea was followed over 4-5 days typically by following corneal thickness via OCT and corneal clarity via bright field imaging (both Phoenix micron IV) (FIGS. 4 and 5A and 5B).

### Example XIII. Methods for Cornea and Conjunctival Organoids

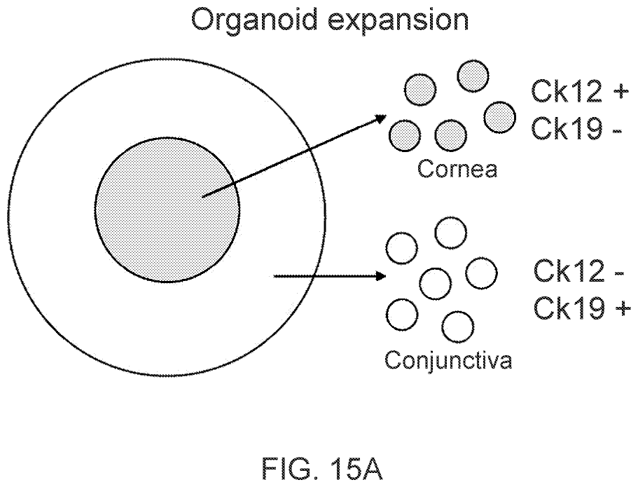

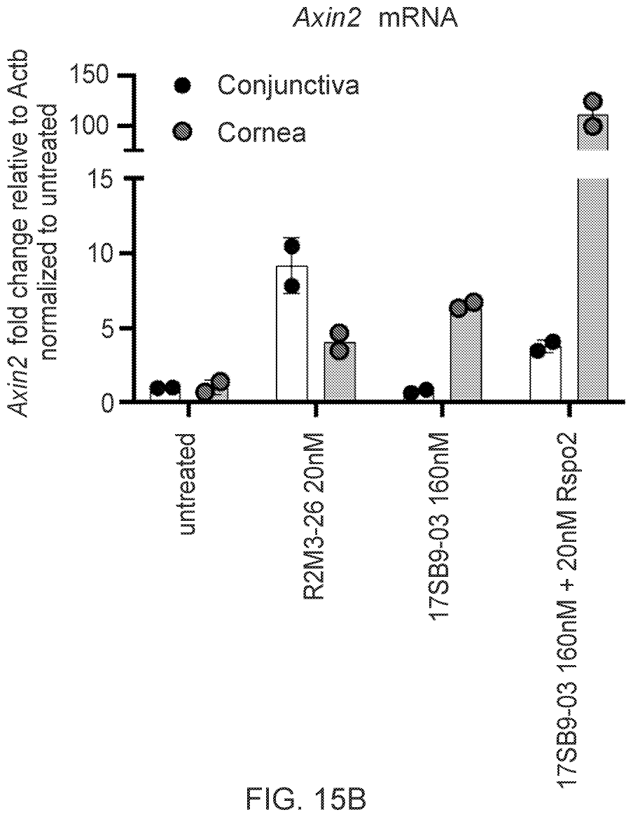

An average of three mice (6 eyeballs) were used per experiment. Eyes were enucleated and cornea and conjunctiva separated under a dissection scope as diagrammed in FIG. 15A. Cornea epithelium was digested to a single cell suspension in 1× TrypLE Express. The conjunctiva was digested in 2.5 mg/mL collagenase A in HBSS for one hour. Near single cell suspensions were resuspended in Matrigel and plated in 40 uL droplets to solidify. Basal medium contained Advanced DMEM, 10 mM HEPES, 1× pen/strep, 100 mg/mL Normocin, 2 mM GlutaMAX, 1× B27, 1 mM N-acetylcysteine, 100 ng/mL KGF and 10 uM Y-27632. Cells were expanded for 5-7 days prior to 48h treatment with the indicated WNT mimetics. Results are shown in FIG. 15B.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

1) Sudi Patel, Larysa Tutchenko. The refractive index of the human cornea: A review. Cont Lens Anterior Eye 2019 October; 42(5):575-580.
2) Zhang Y, Yeh L K, Zhang S, Call M, Yuan Y, Yasunaga M, Kao W W, Liu C Y. Wnt/beta-catenin signaling modulates corneal epithelium stratification via inhibition of Bmp4 during mouse development. Development. 2015; 142:3383-3393.
3) A Wodarz, R Nusse. Mechanisms of Wnt signaling in development. Annu Rev Cell Dev Biol 1998; 14:59-88.
4) Roel Nusse Wnt signaling in disease and in development Cell Res. 2005 January; 15(1):28-32.
5) Eleonora Maurizi, Davide Schiroli, Roberta Zini, Anna Limongelli, Raffaela Mistó, Claudio Macaluso, Graziella Pellegrini. A fine-tuned O-catenin regulation during proliferation of corneal endothelial cells revealed using proteomics analysis. Sci Rep 2020 August 14; 10(1):13841.
6) Martin N. Nakatsu, Zhenhua Ding, Madelena Y. Ng, Thuy T. Truong, Fei Yu, and Sophie X. Deng Wnt/β-Catenin Signaling Regulates Proliferation of Human Cornea Epithelial Stem/Progenitor Cells. Invest Ophthalmol Vis Sci. 2011 June; 52(7): 4734-4741.
7) Shikha Chawla, Sourabh Ghosh. Establishment of in vitro model of corneal scar pathophysiology. Journal of cellular Physiology. 28 Jun. 2017. https://doi.org/10.1002/jcp.26071

1. A method of treating a keratopathy in a subject, comprising administering an engineered WNT signaling modulator to the subject, wherein the modulator is an engineered WNT agonist or an engineered WNT antagonist.

2.-3. (canceled)

4. The method of claim 1, wherein the engineered WNT agonist is selected from: (i) a WNT mimetic; or (ii) an R-spondin mimetic; or (iii) a super SWAP/super agonist.

5. The method of claim 1, wherein the WNT mimetic is a SWAP™ compound and wherein the R-spondin mimetic is a SWEETS™ compound; or a WNT enhancer superSWAP compound.

6. The method of claim 1, wherein the WNT signaling modulator is any of WNT3a or any homologue, mutant, fragment, mimic of WNT3a, or a combination thereof.

7. The method of claim 1, wherein the WNT signaling modulator is co-administered with R-spondin-1, or any R-spondin-1 homologue, mutant, fragment, mimic, or any combination thereof.

8. The method of claim 1, wherein the WNT signaling modulator is administered to the subject in a therapeutically effective dose, wherein the subject is a human patient.

9. (canceled)

10. The method of claim 1, wherein the WNT signaling modulator is administered via eye drop, intracameral injection, intravitreal injection, intrastromal or subconjunctival injection.

11. The method of claim 1, wherein the WNT signaling modulator is suspended in aqueous solution at a concentration between 0.1 nM and 100 mM.

12.-13. (canceled)

14. The method of claim 1, wherein the engineered WNT agonist and engineered WNT antagonist comprise binding compositions that bind to one or more Fzd receptors and binding compositions that bind to one or more LRP receptors.

15. (canceled)

16. The method of claim 14, wherein the FZD binding composition of the engineered WNT agonist are selected from the group consisting of Fzd1, Fzd2, and Fzd7.

17. The method of claim 1, comprising administering an engineered WNT agonist and an engineered WNT antagonist, wherein the engineered WNT agonist and engineered WNT antagonist are administered independently, sequentially, or co-administered at early and/or late stages of the keratopathy.

18.-19. (canceled)

20. The method of claim 1, comprising administering an isolated polynucleotide encoding a polypeptide of the Wnt signaling modulator, optionally wherein the polynucleotide is an mRNA, optionally a modified mRNA.

21. The method of claim 20, comprising administering an expression vector comprising the isolated polynucleotide.

22. (canceled)

23. The method of claim 1, wherein the keratopathy is selected from the group consisting of: bacterial infections, viral infections, fungal infections, protozoan infections, archaeal infections, genetic disorders, brittle cornea syndrome (BCS), corneal endothelial-mesenchymal transition (EnMT), corneal epithelial-mesenchymal transition (EMT), corneal fibrosis, Cogan syndrome, corneal ulcer, epithelial basement membrane dystrophy (EBMD), Fleck corneal dystrophy, Fuchs' dystrophy, post-cornea transplant scarring, gelatinous droplike corneal dystrophy, granular corneal dystrophy type I, granular corneal dystrophy type IL, interstitial keratitis, iridocorneal endothelial syndrome (ICE), keratoconjunctivitis sicca, keratoconus, keratomalacia, lattice dystrophy type I, lattice dystrophy type II, Lisch corneal dystrophy, macular corneal dystrophy, Meesmann corneal dystrophy, bullous keratopathy, aniridia, peripheral ulcerative keratitis, phlyctenular keratoconjunctivitis, posterior polymorphous corneal dystrophy, pterygium, Reis-Buckler corneal dystrophy, Schnyder crystalline corneal dystrophy, Stevens-Johnson syndrome, superficial punctate keratitis, Thiel-Behnke corneal dystrophy, neurotrophic keratitis, herpetic keratitis, scarring, limbal stem cell deficiency and any combination thereof.

24.-25. (canceled)

26. The method of claims 1-24, wherein the keratopathy is post-cornea-transplant scarring.

27. A method of increasing cell yield of mammalian cornea epithelium and/or endothelium cells grown ex vivo prior or post cornea or corneal cell transplant, comprising: treating a mammalian donor cornea or isolated mammalian cornea cells with an engineered WNT signaling modulator, wherein the modulator is an engineered WNT agonist or an engineered WNT antagonist.

28.-29. (canceled)

30. The method of claim 27, wherein the engineered WNT agonist is selected from: (i) WNT3a; (ii) a WNT mimetic; (iii) an R-spondin mimetic; or (iv) a WNT mimetic fused to a mutant R-spondin mimetic referred to as a superSWAP or superagonist.

31. The method of claim 30 wherein the WNT mimetic is a SWAP™ compound and wherein the R-spondin mimetic is a SWEETS™ compound.

32. The method of claim 27, wherein the WNT signaling modulator is any of:
WNT3a or any homologue, mutant, fragment, mimic of WNT3a;
G211-18R5;
R2M3-26;
1SH1-03;
hp1SH1-03
17SB9-03;
SuperSWAP or Superagonist; or
a combination thereof.

33. The method of claim 32 wherein the WNT signaling modulator is co-administered with R-spondin-1, or any R-spondin-1 homologue, mutant, fragment, mimic, or any combination thereof.

34.-42. (canceled)


SEQUENCE LISTING (16 sequences)

SEQ ID NO: 1 - protein, 335 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSDIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 2 - protein, 589 aa, synthetic construct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 340 aa, synthetic construct
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGSGSGQAVVLQEPSLSVSPGGTVTLTCGLSSGSVSTNYYPSWYQQTPGQAPRTLIYYTNTRSSDVPERFSGSIVGNKAALTITGAQPDDESVYFCLLYLGRGIWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - protein, 452 aa, synthetic construct
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCASSKEKATYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 337 aa, synthetic construct
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGSGSGSYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - protein, 454 aa, synthetic construct
QVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 341 aa, synthetic construct
EVQLVESGGGLVQPGGSLRLSCASSANIQSIETLGWYRQAPGKQRELIANMRGGGYMKYADSLKGRFTMSTDNSKNTMYLQMNSLRAEDTAVYYCYVKLRDEDYVYRGQGTQVTVSSGGGGSDIVMTQSPLSLPVTPGEPASISCRSSQSLLHSGGYTYLDWYLQKPGQSPQLLIYLGSKRASGVPDRFSGSGSGTDFTLKISRVEAEDVGVYYCMQALQIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 452 aa, synthetic construct
EVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARHAGFYGLADYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPVLDSDGSFFLYSKLTVDKSRWQQGNVFSCSVMHEALHNHYTQKSLSLSPGK

SEQ ID NO: 9 - protein, 341 aa, synthetic construct
DVQLVESGGGLVQPGGSLRLSCTSSANINSIETLGWYRQAPGKQRELIANMRGGGYMKYAGSLKGRFTMSTESAKNTMYLQMNSLKPEDTAVYYCYVKLRDDDYVYRGQGTQVTVSSGGSGSDVVMTQSPLSLPVTPGEPASISCRSSQSLLHSNGYNYLDWYLQKPGQSPQLLIYLGSKRASGVPDRFSGSGSGTDFTLKISRVEAEDVGVYYCMQALQIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 453 aa, synthetic construct
EVQLVESGPGLVKPSQTLSLTCTVSGGSISSGGYYWSWIRQHPGKGLEWIGYIYYSGSTYYNPSLKSRVTISVDTSKNQFSLKLNSVTAADTAVYYCARHAGFYGLADYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 335 aa, synthetic construct; identical to SEQ ID NO: 1

SEQ ID NO: 12 - protein, 711 aa, synthetic construct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGSGGSGGGGSNPICKGCLSCSKDNGCSRCQQKLFFFLRREGMRQYGECLHSCPSGYYGHRAPDMNRCARCRIENCDSCRSKDACTKCKVGFYLHRGRCFDECPDGFAPLEETMECVE

SEQ ID NO: 13 - protein, 338 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWYGVAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 712 aa, synthetic construct
RAEVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVESGGGLVQPGGSLRLSCAASGFNISSSYIHWVRQAPGKGLEWVAYIYSSYGSTYYADSVKGRFTISADTSKNTAYLQMNSLRAEDTAVYYCARASWYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGSGGSGGGGSNPICKGCLSCSKDNGCSRCQQKLFFFLRREGMRQYGECLHSCPSGYYGHRAPDMNRCARCRIENCDSCRSKDACTKCKVGFYLHRGRCFDECPDGFAPLEETMECVE

SEQ ID NO: 15 - protein, 476 aa, synthetic construct
DVQLVESGGGLVQPGGSLRLSCTSSANINSIETLGWYRQAPGKQRELIANMRGGGYMKYAGSLKGRFTMSTESAKNTMYLQMNSLKPEDTAVYYCYVKLRDDDYVYRGQGTQVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGSAVQQVESGGGLVQPGGSLRLSCAASGFTLDYYAISWFRQAPGKKRELVADITSGGSTNYADSVKGRFTISRDNAKNTIYLQMNSLKPEDTAVYFCNAVTYNGYTIWGQGTQVTVSSGSGSGHHHHHH

SEQ ID NO: 16 - protein, 478 aa, synthetic construct
AVQQVESGGGLVQPGGSLRLSCAASGFTLDYYAISWFRQAPGKKRELVADITSGGSTNYADSVKGRFTISRDNAKNTIYLQMNSLKPEDTAVYFCNAVTYNGYTIWGQGTQVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGSDVQLVESGGGLVQPGGSLRLSCTSSANINSIETLGWYRQAPGKQRELIANMRGGGYMKYAGSLKGRFTMSTESAKNTMYLQMNSLKPEDTAVYYCYVKLRDDDYVYRGQGTQVTVSSGSGSGDYKDDDDK